(19) United States
(12) Patent Application Publication
(10) Pub. No.: US 2022/0354836 A1
(43) Pub. Date: Nov. 10, 2022

(54) Equine Esomeprazole Formulations and Methods of Use

(71) Applicant: Kindred Biosciences, Inc., Burlingame, CA (US)

(72) Inventors: Geeta Srivastava, San Diego, CA (US); Om P. Srivastava, San Diego, CA (US); Emily Sundman, Lipan, TX (US); Melinda Poole O'Banion, Louisburg, KS (US)

(73) Assignee: Kindred Biosciences, Inc., Burlingame, CA (US)

(21) Appl. No.: 17/626,936

(22) PCT Filed: Jul. 15, 2020
(86) PCT No.: PCT/US2020/042127

§ 371 Date: Jan. 13, 2022

Related U.S. Application Data
(60) Provisional application No. 63/038,549, filed on Jun. 12, 2020.
(60) Provisional application No. 62/874,702, filed on Jul. 16, 2019.

(51) Int. Cl. A61K 31/4439; A61K 9/00; A61K 47/44; A61K 47/14; A61K 47/02; A61P 1/04

(52) U.S. Cl. CPC A61K 31/4439; A61K 9/0019; A61K 47/44; A61K 47/14; A61K 47/02; A61P 1/04

(57) ABSTRACT

The present disclosure relates to formulations of proton pump inhibitors such as omeprazole or esomeprazole including their salts or hydrated salts, such as esomeprazole sodium, esomeprazole magnesium or esomeprazole magnesium dihydrate, and methods of use in treating ulcers in equines and other veterinary animals The disclosure also relates to injectable formulations of proton pump inhibitors such as omeprazole or esomeprazole including their salts or hydrated salts, such as esomeprazole sodium, esomeprazole magnesium, or esomeprazole magnesium dihydrate, which have a small particle size, such as a median particle size of less than 10 μm or less than 5 μm.

### FIELD

The present disclosure relates to formulations of proton pump inhibitors such as omeprazole or esomeprazole, such as esomeprazole magnesium or esomeprazole magnesium dihydrate, and methods of use in treating gastric ulcers in equines and other veterinary animals. The disclosure also relates to injectable formulations of proton pump inhibitors, such as omeprazole or esomeprazole, for example, esomeprazole magnesium, such as esomeprazole magnesium dihydrate, which have a small particle size, such as a median particle size of less than 10 μm or less than 5 μm.

### BACKGROUND

Modern domesticated equines suffer from gastric ulcers at relatively high rates, and the symptoms of ulcers, such as stomach and gastrointestinal pain and upset, can interfere with the wellbeing of the animal as well as, in the case of an equine used for sport, the animal's work and training. It is possible that the relatively high rates of gastric ulcers seen in domesticated equines are due to the stabling and feeding patterns associated with domestication, for example, in which equines are fed at several specific points during a day rather than allowed to constantly forage for food as they would in the wild. These different feeding conditions might impact the function and acidity of the stomach and upper intestines, possibly making gastric ulcers more common.

Equines are frequently administered oral proton pump inhibitor pastes if they have been diagnosed with gastric ulcers, for example, by endoscopy, or if they display symptoms of ulcers such as discomfort, loss of condition, and irritability or anxiety with training, or are subject to stressful conditions such as transport that may provoke ulcer-like symptoms or development of ulceration. Common oral proton pump inhibitor pastes for equines are the omeprazole pastes sold under the trade names Gastrogard® and Ulcergard®. These oral paste compositions, while effective, are expensive, difficult to administer accurately as the horse must tolerate and ingest a complete dose, require that the animal can receive oral medications (which may not be possible in certain settings), typically require daily administration, and can be messy and difficult to handle and store.

The present disclosure relates to alternative, injectable formulations of proton pump inhibitors such as omeprazole or esomeprazole (including their salts or hydrated salts), such as esomeprazole magnesium, that, in contrast, can be relatively simple to administer, and accordingly do not rely on oral administration, which may improve the animal's compliance with the administration, and which may be suitable for equines that are unable to receive oral medications. Furthermore, the formulations herein do not necessarily need to be given daily as their bioavailability is such that they may reduce stomach acid levels, and thus promote healing of damaged mucosal tissue, for a longer time between doses than an oral paste. Accordingly, with the formulations herein, an equine may, in some embodiments, be injected only, for example, once or twice weekly rather than having to tolerate a daily oral paste administration.

The specific, injectable pharmaceutical formulations herein also provide specific mixtures of proton pump inhibitor and excipients that allow for long-term storage and ease of administration to equines and other veterinary animals as well as simple and less frequent administration procedures.

### SUMMARY

The present disclosure includes, for example, pharmaceutical formulations comprising a suspension of a proton pump inhibitor such as omeprazole or esomeprazole, such as esomeprazole sodium, esomeprazole magnesium, omeprazole magnesium, omeprazole sodium, or another omeprazole or esomeprazole salt or hydrated salt in a mixture of a plant oil and caprylic/capric triglyceride. In some embodiments, the pharmaceutical formulation comprises a suspension of esomeprazole in a mixture of a plant oil and caprylic/capric triglyceride. In some embodiments, the pharmaceutical formulation comprises a suspension of esomeprazole magnesium salt (e.g. esomeprazole magnesium dihydrate) in a mixture of a plant oil and caprylic/capric triglyceride. In some embodiments, the pharmaceutical formulation comprises a suspension of omeprazole (e.g. omeprazole magnesium) in a mixture of a plant oil and caprylic/capric triglyceride. The plant oil may be selected from the group consisting of: canola oil, coconut oil, corn oil, cottonseed oil, olive oil, palm oil, peanut oil, safflower oil, sesame oil, soybean oil, and sunflower oil, and mixtures of any two or more of canola oil, coconut oil, corn oil, cottonseed oil, olive oil, palm oil, peanut oil, safflower oil, sesame oil, soybean oil, and sunflower oil. In some embodiments, the plant oil is cottonseed oil or a mixture of cottonseed oil with another plant oil. In some embodiments, the plant oil is cottonseed oil.

In some embodiments, the formulation comprises 15% to 25% weight/weight (w/w) esomeprazole magnesium, such as 18% to 22% w/w esomeprazole magnesium, 19% to 21% w/w esomeprazole magnesium, or 20% w/w omeprazole or esomeprazole, such as esomeprazole sodium, esomeprazole magnesium, or another omeprazole or esomeprazole salt or hydrated salt. In some embodiments, the formulation comprises 15% to 25% weight/weight (w/w) esomeprazole magnesium, such as 18% to 22% w/w esomeprazole magnesium, 19% to 21% w/w esomeprazole magnesium, or 20% w/w esomeprazole magnesium. In some embodiments, the esomeprazole magnesium is esomeprazole magnesium dihydrate. In some embodiments, the formulation comprises 15% to 25% weight/weight (w/w) omeprazole magnesium, such as 18% to 22% w/w omeprazole magnesium, 19% to 21% w/w omeprazole magnesium, or 20% w/w omeprazole magnesium. In some embodiments, the formulation comprises 15% to 25% weight/weight (w/w) omeprazole magnesium, such as 18% to 22% w/w omeprazole magnesium, 19% to 21% w/w omeprazole magnesium, or 20% w/w omeprazole magnesium.

In some embodiments, the formulation comprises 5% to 30% w/w plant oil, such as 5-10%, 10-15%, 15-20%, 20-25%, or 25-30%. In some embodiments, the formulation comprises 5% to 30% w/w cottonseed oil, such as 5-10%, 10-15%, 15-20%, 20-25%, or 25-30%. In some embodiments, the formulation comprises 10-15%, 10-12%, 13-15%, 15-17%, 18-20%, or 15-20% w/w cottonseed oil. In some embodiments, the formulation comprises 50% to 90% w/w caprylic/capric triglyceride, such as 50-60%, 60-70%, 70-80%, or 80-90%. In some embodiments, the formulation comprises 60-70%, 60-65%, 65-70%, 70-80%, 70-75%, or 75-80% w/w caprylic/capric triglyceride.

The formulations herein may also contain at least one preservative such as butylated hydroxytoluene (BHT) or butylated hydroxyanisole (BHA) or sodium bisulfate, or a mixture of two or more preservatives. The preservative may comprise a single additional ingredient or more than one additional ingredient. In some embodiments, the preservative comprises butylated hydroxytoluene (BHT). In some embodiments, the preservative comprises 0.05-1.0% w/w BHT. In some embodiments, the preservative comprises 0.05-0.15% w/w BHT. In some embodiments, the preservative comprises 0.1% w/w BHT. In some embodiments, the preservative comprises 0.1-1.0%, 0.1-0.2%, 0.1-0.5%, 0.5-1.0%, 0.05%, 0.08%, 0.09%, 0.1%, 0.11%, 0.12%, 0.13%, 0.14%, 0.15%, 0.2%, 0.3%, 0.4%, 0.5%, 0.6%, 0.7%, 0.8%, 0.9%, or 1.0% w/w BHT or BHA.

In some cases herein, formulations may consist essentially of esomeprazole magnesium, cottonseed oil, caprylic/capric triglyceride, and at least one preservative. In some such cases, the formulation consists essentially of the proton pump inhibitor such as omeprazole or esomeprazole, such as esomeprazole sodium, esomeprazole magnesium, or another omeprazole or esomeprazole salt or hydrated salt, cottonseed oil, caprylic/capric triglyceride, and at least one preservative such as BHT. In some such cases, the formulation consists essentially of esomeprazole magnesium, cottonseed oil, caprylic/capric triglyceride, and BHT. In some cases herein, formulations may consist essentially of esomeprazole magnesium dihydrate, cottonseed oil, caprylic/capric triglyceride, and at least one preservative. In some such cases, the formulation consists essentially of esomeprazole magnesium dihydrate, cottonseed oil, caprylic/capric triglyceride, and BHT.

Formulations herein also include injectable formulations comprising a suspension of 18-22% weight/weight (w/w) proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium in a mixture of cottonseed oil and caprylic/capric triglyceride, and 0.05-1.0% w/w preservative, as well as formulations consisting essentially of a suspension of 18-22% weight/weight (w/w) proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium in a mixture of cottonseed oil and caprylic/capric triglyceride, and 0.05-1.0% w/w preservative. In some embodiments, the esomeprazole magnesium is esomeprazole magnesium dihydrate. In some embodiments, the formulation comprises 5-30% cottonseed oil, such as 5-25%, 10-15%, 10-12%, 13-15%, 15-17%, 18-20%, 15-20%, 20-22%, or 20-25% w/w cottonseed oil. In some embodiments, the formulation comprises 50% to 90% w/w caprylic/capric triglyceride, such as 60-70%, 60-65%, 65-70%, 70-80%, 70-75%, 75-80%, 80-90%, 80-85%, or 85-90%.

In any of the exemplary formulations herein, the caprylic/capric triglyceride may comprise 50-65% caprylic acid and 30-45% capric acid, or may be Miglyol® 812. In any of the exemplary formulations herein, the caprylic/capric triglyceride may comprise 50-75% caprylic acid and 22-45% capric acid, or may be Captex® 355. The caprylic/capric triglyceride may be derived from plant oil sources and may contain other ingredients such as glycerin, for example, or small amounts of longer chain fatty acids.

In some of the above formulation embodiments, the formulation has a median particle size of less than 10 μm, such as less than 8 μm, less than 5 μm, less than 4 μm, or less than 2.5 μm. In any of the embodiments above, the formulation as described above may have at least one of the following properties:
a) the formulation is suitable for intramuscular injection to an equine or other animal;
b) the formulation is suitable for subcutaneous injection to an equine or other animal;
c) after 6 months of storage at 2-8, 25, or 30° C., the median particle size of the formulation remains less than 8 μm;
d) after 6 months of storage at 2-8, 25, or 30° C., the median particle size of the formulation remains less than 5 μm;
e) after 6 months of storage at 2-8, 25, or 30° C., the median particle size of the formulation remains less than 4 μm;
f) after 6 months of storage at 2-8, 25, or 30° C., the median particle size of the formulation remains less than 2.5 μm;
g) after 6 months of storage at 2-8, 25, or 30° C., the particle size of the 90^{th} percentile of the particle size distribution curve remains less than 8 μm;
h) there is no significant change in the viscosity of the formulation after 6 months storage at 2-8, 25, or 30° C.;
i) viscosity remains below 300 cP, between 200 and 300 cP, between 225 and 300 cP, or between 250 and 300 cP after 6 months of storage at 2-8, 25, or 30° C.;
j) there is no change in the injectability of the formulation after 6 months at room temperature (e.g. 25° C.) using a 3 mL 21 G×½″ syringe/needle;
k) total proton pump inhibitor impurities remain less than 0.15% after up to 6 months of storage at 2-8, 25, or 30° C.;
l) after 12 months of storage at 2-8 or 25° C., the median particle size of the formulation remains less than 8 μm;
m) after 12 months of storage at 2-8 or 25° C., the median particle size of the formulation remains less than 5 μm;
n) after 12 months of storage at 2-8 or 25° C., the median particle size of the formulation remains less than 4 μm;
o) after 12 months of storage at 2-8 or 25° C., the median particle size of the formulation remains less than 2.5 μm;
p) after 12 months of storage at 2-8 or 25° C., the particle size of the 90^{th} percentile of the particle size distribution curve remains less than 8 μm;
q) there is no significant change in the viscosity of the formulation after 12 months storage at 2-8 or 25° C.;
r) viscosity remains below 300 cP, between 200 and 300 cP, between 225 and 300 cP, or between 250 and 300 cP after 12 months of storage at 2-8 or 25° C.;
s) there is no change in the injectability of the formulation after 12 months at room temperature (e.g. 25° C.) using a 3 mL 21 G×½″ to 18 G×½″ syringe/needle;
t) total proton pump inhibitor impurities remain less than 0.15% after up to 12 months of storage at 2-8 or 25° C.; and/or
u) the mean half-life (T_{1/2}) of the formulation when injected intramuscularly to horses ranges from 12 to 24 hours.

In some embodiments, after 24 months at 2-8° C. or 25° C. or 30° C., the median particle size of the formulation remains less than 10 μm, less than 8 μm, less than 5 μm, less than 4 μm, or less than 2.5 μm. In some embodiments, after 24 months at 2-8° C. or 25° C. or 30° C., the formulation remains stable for use.

Additional embodiments herein comprise injectable pharmaceutical formulations comprising a suspension of proton pump inhibitor such as omeprazole or esomeprazole, such as esomeprazole sodium, esomeprazole magnesium, or another omeprazole or esomeprazole salt or hydrated salt, such as esomeprazole magnesium dihydrate or such as omeprazole magnesium or omeprazole sodium in an oil comprising medium-chain to long-chain fatty acids, such as medium-chain to long-chain triglycerides. In some embodiments, the median particle size of the formulations is less than 10 μm, less than 8 μm, less than 5 μm, less than 4 μm, or less than 2.5 μm. In other cases, the median particle size may be larger than 10 μm, larger than 8 μm, larger than 5 μm, larger than 4 μm, or larger than 2.5 μm. In some cases, the proton pump inhibitor, either prior to or after being formulated, is micronized so that the formulation will achieve a median particle size of less than 10 μm, less than 8 μm, less than 5 μm, less than 4 μm, or less than 2.5 μm. In other cases, micronization is not necessary to achieve this particle size and is, therefore, not performed. In some cases where micronization is performed, after six months, the micronization to achieve a median particle size of less than 10 μm, less than 8 μm, less than 5 μm, less than 4 μm, or less than 2.5 μm does not affect the stability of the formulation. In other cases, micronization may improve the stability of the formulation. In cases where micronization does not improve the stability of the formulation, it may not be performed.

In some embodiments, the medium-chain to long-chain fatty acids are long-chain triglycerides. In some embodiments, the medium-chain to long-chain fatty acids are a mixture of medium-chain to long-chain triglycerides. In some embodiments, the medium-chain to long-chain triglycerides comprise plant oil. In some embodiments, the plant oil selected from the group consisting of: canola oil, coconut oil, corn oil, cottonseed oil, olive oil, palm oil, peanut oil, safflower oil, sesame oil, soybean oil, and sunflower oil, and mixtures of any two or more of canola oil, coconut oil, corn oil, cottonseed oil, olive oil, palm oil, peanut oil, safflower oil, sesame oil, soybean oil, and sunflower oil. In some embodiments, the plant oil is cottonseed oil or a mixture of cottonseed oil with another plant oil. In some embodiments, the medium-chain to long-chain triglycerides comprise caprylic/capric triglyceride. In some embodiments, the caprylic/capric triglyceride comprises 50-65% caprylic acid and 30-45% capric acid, or wherein the caprylic/capric triglyceride comprises Miglyol® 812. In some embodiments, the caprylic/capric triglyceride comprises 50-75% caprylic acid and 22-45% capric acid, or wherein the caprylic/capric triglyceride comprises Captex® 355.

In some embodiments, the above formulation comprises 50% to 90% w/w caprylic/capric triglyceride, such as 50-60%, 60-70%, 70-80%, or 80-90%. In some embodiments, the formulation comprises 60-70%, 60-65%, 65-70%, 70-80%, 70-75%, or 75-80% w/w caprylic/capric triglyceride. In some embodiments, the formulation comprises 15% to 25% weight/weight (w/w) esomeprazole magnesium dihydrate. In some embodiments, the formulation comprises 18% to 22% w/w esomeprazole magnesium. In some embodiments, the formulation comprises 19% to 21% w/w esomeprazole magnesium. In some embodiments, the formulation comprises 20% w/w esomeprazole magnesium. In some embodiments, the formulation comprises 5% to 30% w/w plant oil, such as 5-10%, 10-15%, 15-20%, 20-25%, or 25-30%. In some embodiments, the formulation comprises 5% to 30% w/w cottonseed oil, such as 5-10%, 10-15%, 15-20%, 20-25%, or 25-30%. In some embodiments, the formulation comprises 10-15%, 10-12%, 13-15%, 15-17%, 18-20%, or 15-20% w/w cottonseed oil. In some embodiments, the formulation comprises 5-30% cottonseed oil, such as 5-25%, 10-15%, 10-12%, 13-15%, 15-17%, 18-20%, 15-20%, 20-22%, or 20-25% w/w cottonseed oil. In some embodiments, the formulation comprises 50% to 90% w/w caprylic/capric triglyceride, such as 60-70%, 60-65%, 65-70%, 70-80%, 70-75%, 75-80%, 80-90%, 80-85%, or 85-90%.

The formulations herein may also contain at least one preservative such as butylated hydroxytoluene (BHT), butylated hydroxyanisole (BHA), sodium bisulfate, vitamin C, a bacterial growth inhibitor such as sodium nitrile, sulfur dioxide, benzoic acid, methylparaben, propylparaben, benzyl alcohol, phenol, a cresol such as m-cresol, chlorobutanol, or a mixture of two or more preservatives. The preservative may comprise a single additional ingredient or more than one additional ingredient. In some embodiments, the preservative comprises butylated hydroxytoluene (BHT). In some embodiments, the preservative is present in an amount of 0.05-1.0% w/w. In some embodiments, the preservative is present at 0.05-0.15% w/w. In some embodiments, the preservative is present at 0.1% w/w. In some embodiments, the preservative is present at 0.1-1.0%, 0.1-0.2%, 0.1-0.5%, 0.5-1.0%, 0.05%, 0.08%, 0.09%, 0.1%, 0.11%, 0.12%, 0.13%, 0.14%, 0.15%, 0.2%, 0.3%, 0.4%, 0.5%, 0.6%, 0.7%, 0.8%, 0.9%, or 1.0% w/w. In some embodiments, the preservative comprises 0.05-1.0% w/w BHT. In some embodiments, the preservative comprises 0.05-0.15% w/w BHT. In some embodiments, the preservative comprises 0.1% w/w BHT. In some embodiments, the preservative comprises 0.1-1.0%, 0.1-0.2%, 0.1-0.5%, 0.5-1.0%, 0.05%, 0.08%, 0.09%, 0.1%, 0.11%, 0.12%, 0.13%, 0.14%, 0.15%, 0.2%, 0.3%, 0.4%, 0.5%, 0.6%, 0.7%, 0.8%, 0.9%, or 1.0% w/w BHT or BHA.

In some embodiments, the formulation consists essentially of omeprazole or esomeprazole, such as esomeprazole sodium, esomeprazole magnesium, or another omeprazole or esomeprazole salt or hydrated salt, medium-chain to long-chain triglycerides, and preservative. In some embodiments, the formulation consists essentially of esomeprazole magnesium dihydrate, medium-chain to long-chain triglycerides, and preservative. In some such embodiments, the formulation has at least one of the following properties:
a) the formulation is suitable for intramuscular injection to an equine or other animal;
b) the formulation is suitable for subcutaneous injection to an equine or other animal;
c) after 6 months of storage at 2-8, 25, or 30° C., the median particle size of the formulation remains less than 8 μm;
d) after 6 months of storage at 2-8, 25, or 30° C., the median particle size of the formulation remains less than 5 μm;
e) after 6 months of storage at 2-8, 25, or 30° C., the median particle size of the formulation remains less than 4 μm;
f) after 6 months of storage at 2-8, 25, or 30° C., the median particle size of the formulation remains less than 2.5 μm;
g) after 6 months of storage at 2-8, 25, or 30° C., the particle size of the 90^{th} percentile of the particle size distribution curve remains less than 8 μm;
h) there is no significant change in the viscosity of the formulation after 6 months storage at 2-8, 25, or 30° C.;
i) viscosity remains below 300 cP, between 200 and 300 cP, between 225 and 300 cP, or between 250 and 300 cP after 6 months of storage at 2-8, 25, or 30° C.;
j) there is no change in the injectability of the formulation after 6 months at room temperature (e.g. 25° C.) using a 3 mL 21 G×½″ syringe/needle;
k) total proton pump inhibitor impurities remain less than 0.15% after up to 6 months of storage at 2-8, 25, or 30° C.; 1) after 12 months of storage at 2-8 or 25° C., the median particle size of the formulation remains less than 8 μm;
m) after 12 months of storage at 2-8 or 25° C., the median particle size of the formulation remains less than 5 μm;
n) after 12 months of storage at 2-8 or 25° C., the median particle size of the formulation remains less than 4 μm;
o) after 12 months of storage at 2-8 or 25° C., the median particle size of the formulation remains less than 2.5 μm;
p) after 12 months of storage at 2-8 or 25° C., the particle size of the 90^{th} percentile of the particle size distribution curve remains less than 8 μm;
q) there is no significant change in the viscosity of the formulation after 12 months storage at 2-8 or 25° C.;
r) viscosity remains below 300 cP, between 200 and 300 cP, between 225 and 300 cP, or between 250 and 300 cP after 12 months of storage at 2-8 or 25° C.;
s) there is no change in the injectability of the formulation after 12 months at room temperature (e.g. 25° C.) using a 3 mL 21 G×½″ to 18 G×½″ syringe/needle;
t) total proton pump inhibitor impurities remain less than 0.15% after up to 12 months of storage at 2-8 or 25° C.; and/or
u) the mean half-life (T_{1/2}) of the formulation when injected intramuscularly to horses ranges from 12 to 24 hours.

In some embodiments, after 24 months at 2-8° C. or 25° C. or 30° C., the median particle size of the formulation remains less than 10 μm, less than 8 μm, less than 5 μm, less than 4 μm, or less than 2.5 μm. In some embodiments, after 24 months at 2-8° C. or 25° C. or 30° C., the formulation remains stable for use.

The present disclosure also includes vials containing the formulations herein, and which may be suitable for transporting and storing the formulations. Vials herein may be of any shape or type of container and may be configured to allow for easy dispensing of the formulation for administration. The present disclosure also includes devices for intramuscular or subcutaneous injection to equines or other animals for administering the formulations herein. Such devices may contain any of the above formulations. Such devices include, for example, syringes with needles for intramuscular injection or injection pen devices for intramuscular injection. In some embodiments, each device comprises a complete, single unit dosage of esomeprazole magnesium. Thus, each device may be used for a single injection of a unit dosage and then be discarded.

The present disclosure also includes processes for preparing the injectable equine pharmaceutical formulations described above. In some embodiments, the processes comprise (a) mixing the plant oil or cottonseed oil with the caprylic/capric triglyceride, and (b) adding omeprazole or esomeprazole, such as esomeprazole sodium, esomeprazole magnesium, or another omeprazole or esomeprazole salt or hydrated salt such as omeprazole magnesium or omeprazole sodium to the mixture of (a) to create a suspension of the omeprazole or esomeprazole, such as esomeprazole sodium, esomeprazole magnesium, or another omeprazole or esomeprazole salt or hydrated salt such as omeprazole magnesium or omeprazole sodium in the mixture, and optionally adding at least one preservative to the mixture of plant oil or cottonseed oil and caprylic/capric triglyceride and/or to the suspension. In other embodiments, the processes comprise: (a) obtaining and optionally mixing the medium-chain to long-chain triglycerides, and (b) adding omeprazole or esomeprazole, such as esomeprazole sodium, esomeprazole magnesium, or another omeprazole or esomeprazole salt or hydrated salt to the product of (a) to create a suspension of the omeprazole or esomeprazole, such as esomeprazole sodium, esomeprazole magnesium, or another omeprazole or esomeprazole salt or hydrated salt such as omeprazole magnesium or omeprazole sodium in the mixture, and optionally adding at least one preservative to the product of (a) or to the suspension of (b).

The present disclosure also includes uses of the above formulations, for example, for treating gastric ulcers in animals such as equines. In some embodiments, the treatment methods comprise administering an effective amount of a formulation as described above to an animal such as an equine at least once per week by intramuscular or subcutaneous injection. In some embodiments, the formulation is administered so as to provide at least one dose of between 1.5 mg/kg and 5.0 mg/kg esomeprazole magnesium at least once per week. In some embodiments, the formulation is administered once, twice or three times per week. In some embodiments, the formulation is administered at a dose of 1.5 mg/kg, 1.75 mg/kg, 2.0 mg/kg, 2.5 mg/kg, 3.0 mg/kg, 3.5 mg/kg, 4 mg/kg, 4.5 mg/kg, or 5 mg/kg esomeprazole magnesium. In some embodiments, the formulation is administered over a period of 4 weeks. In some embodiments, the formulation is administered over a period of 3 weeks. In some embodiments, the formulation is administered according to one of the following regimens: (a) once per week for 3 weeks at a dose of 2.0-4.0 mg/kg; (b) once per week for 4 weeks at a dose of 2.0-4.0 mg/kg; (c) twice per week for 3 weeks at a dose of 2.0-4.0 mg/kg; (d) twice per week for 4 weeks at a dose of 2.0-4.0 mg/kg; or (e) any one of (a) to (d) followed by a reduced dose once or twice per week for at least one additional week. In some embodiments, the first dose or the first two doses or the first week, two weeks, three weeks, or four weeks of doses are at a higher level (e.g. a “loading dose”) than subsequent doses. In some embodiments, the formulation is administered for a period longer than 4 weeks, optionally wherein the dose is reduced after a period of 4 weeks. In some embodiments, the formulation is administered for a period longer than 3 weeks, optionally wherein the dose is reduced after a period of 3 weeks. In any of the above dosing regimens, the formulation can be administered by intramuscular injection or by subcutaneous injection. In some embodiments, the formulation is administered using the vial and/or the device as described above. In some embodiments, the formulation is administered to an equine. In some embodiments, the equine suffers from gastric ulcer, equine gastric ulcer syndrome (EGUS), equine squamous gastric disease (ESGD), or equine glandular gastric disease (EGGD). In some embodiments, the gastric ulcer is treated, for example, in some embodiments at least one ulcerous lesion is resolved or reduced in size, and/or the number of lesions is reduced.

The present disclosure also includes an injectable pharmaceutical composition for use in the treatment of ulcer in animals such as equines. In some embodiments, the injectable pharmaceutical formulation is for use in a method of treatment of ulcer in an equine, wherein the formulation is administered to the equine by intramuscular or subcutaneous injection. In some embodiments, the formulation is administered so as to provide at least one dose of between 1.5 mg/kg and 5.0 mg/kg esomeprazole magnesium at least once per week. In some embodiments, the formulation is administered once, twice or three times per week. In some embodiments, the formulation is administered once per week. In some embodiments, the formulation is administered twice per week. In some embodiments, the formulation is administered three times per week. In some embodiments, the formulation is administered at a dose of 1.5 mg/kg, 1.75 mg/kg, 2.0 mg/kg, 2.5 mg/kg, 3.0 mg/kg, 3.5 mg/kg, 4 mg/kg, 4.5 mg/kg, or 5 mg/kg esomeprazole magnesium. In some embodiments, the formulation is administered over a period of 4 weeks. In some embodiments, the formulation is administered over a period of 3 weeks. In some embodiments, the formulation is administered according to one of the following regimens: (a) Once per week for 3 weeks at a dose of 2.0-4.0 mg/kg; (b) Once per week for 4 weeks at a dose of 2.0-4.0 mg/kg; (c) Twice per week for 3 weeks at a dose of 2.0-4.0 mg/kg; (d) Twice per week for 4 weeks at a dose of 2.0-4.0 mg/kg; or (e) The regimen of any one of (a) to (d) followed by a reduced dose once or twice per week for at least one additional week. In some embodiments, the formulation is administered for a period longer than 4 weeks, optionally wherein the dose is reduced after a period of 4 weeks. In some embodiments, the formulation is administered for a period longer than 3 weeks, optionally wherein the dose is reduced after a period of 3 weeks. In some embodiments, the formulation is administered by intramuscular injection. In some embodiments, the formulation is administered by subcutaneous injection. In some embodiments, the formulation is administered using the vial and/or device as described herein. In some embodiments, the equine suffers from equine gastric ulcer syndrome (EGUS), equine squamous gastric disease (ESGD), or equine glandular gastric disease (EGGD). In some embodiments, the ulcer is treated, such as by (a) reducing the size of at least one ulcerous lesion or (b) reducing the number of ulcerous lesions.

Additional objects of this disclosure are set forth in part in the description which follows, including in the drawings and claims. It is to be understood that both the foregoing general description and the following detailed description are exemplary and explanatory only and are not restrictive of the claims.

The headings provided herein are not limitations of the various aspects of the disclosure, which can be had by reference to the specification as a whole. Accordingly, the terms defined immediately below are more fully defined by reference to the specification in its entirety.

### BRIEF DESCRIPTION OF THE DRAWINGS

FIGS. 1A, 1B, and 1C show the impact of administration of an exemplary formulation of this disclosure on the gastric pH of 5 horses, as described in the working examples section below. FIG. 1A shows the percentage of time that pH was greater than or equal to 4 just prior to (day 0), at (day 1), or after (days 2-6) intramuscular injection of a 1.75 mg/kg dose of esomeprazole magnesium contained in a formulation as described herein. FIG. 1B shows the percentage of time that pH was greater than or equal to 4 just prior to (day 0), at (day 1), or after (days 2-6) intramuscular injection of a second, 4.0 mg/kg dose of esomeprazole magnesium contained in a formulation as described herein. The 4.0 mg/kg injection was administered about 2 weeks after the first 1.75 mg/kg injection. The numbers below the graphs of FIGS. 1A and 1B represent the numbers given to the 5 horses used in the study. FIG. 1C provides the average of the curves of each of FIGS. 1A and 1B, where “phase I” indicates the 1.75 mg/kg dose shown in FIG. 1A and “phase 2” indicates the 4.0 mg/kg dose shown in FIG. 1B.

### DETAILED DESCRIPTION OF CERTAIN EMBODIMENTS

### I. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated. Units, prefixes, and symbols are denoted in their Systeme International de Unites (SI) accepted form. Numeric ranges are inclusive of the numbers defining the range. Measured values herein, such as weight to weight percentages are understood to be approximate, taking into account significant digits and the error associated with the measurement.

All percentage measurements herein are weight to weight (w/w) percentages unless expressly stated otherwise.

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

Formulations herein may be administered to animals such as equines and other livestock. “Equines” herein include any breed of domesticated horses, from mini-horses and small ponies up to large draft horses, as well as mustangs, other wild horse breeds and zebras. The term also includes equines of any age from foal to elderly. Equines include all domesticated horse breeds, of which there are a large number of varying size and body weight, for example mini-horses, small ponies such as Shetland ponies, larger ponies and cobs such as Welsh ponies, German riding ponies, Haflingers and the like, a wide variety of riding and sports horses such as Arabians, Thoroughbreds, Quarter Horses, various types of Warmbloods, as well as Andalusians, Lusitanos, Appaloosas, Standardbreds, etc., draft breeds such as Friesians, Belgians, Clydesdales, and the like, and crosses of the above breeds with other breeds. Other non-equine animals such as donkeys, mules, pigs, llamas, and alpacas may also be treated with the formulations herein. An animal being treated herein may, in some instances, be referred to as a “subject” or “patient.”

A “proton pump inhibitor” refers to a compound that suppresses stomach acid secretion by inhibiting the gastric proton pump H+/K+ATPase. Proton pump inhibitors include, for example, omeprazole, esomeprazole, rabeprazole, lansoprazole, tenatoprazole, including, for example, their pharmaceutically effective stereoisomers such as enantiomers, including salt and hydrate forms. For example, omeprazole, esomeprazole, rabeprazole, lansoprazole, and tenatoprazole may be found as sodium salts or as magnesium salts, in some cases as hydrated magnesium salts.

“Omeprazole” refers to a compound with the molecular formula C17H18N303S, and is a racemic mixture of S and R enantiomers. The term includes any form of omeprazole, such as any salt or hydrate form. “Esomeprazole” refers to the S enantiomer of omeprazole and has the molecular formula Cl7H18N303S. Esomeprazole may be found in various salt or hydrate forms, including esomeprazole sodium, esomeprazole strontium, and esomeprazole magnesium. “Esomeprazole magnesium,” when used generally herein, refers to both esomeprazole magnesium dihydrate and esomeprazole magnesium trihydrate and mixtures of these two hydrate forms as well as anhydrous and monohydrated esomeprazole magnesium. Esomeprazole magnesium trihydrate has a molecular weight of 767.2 g/mol and a molecular formula of C34H42MgN6O9S2, while esomeprazole magnesium dihydrate has a molecular weight of 749.2 g/mol and a molecular formula of C34H40MgN6O8S2. The anhydrous weight of esomeprazole magnesium is 713 g/mol. In these forms, one magnesium ion coordinates two esomeprazole molecules. The terms “esomeprazole magnesium dihydrate” or “esomeprazole magnesium trihydrate” are used to refer to the specific hydrate forms. Those hydrate forms may each contain small amounts of other hydrate forms as impurities, for example. For instance, once an esomeprazole magnesium dihydrate is added to make a suspension for a pharmaceutical formulation, it is possible that its hydrated form is modified somewhat due to exposure to other excipients in the formulation. Other forms of esomeprazole include “esomeprazole sodium,” which has a molecular weight of 367.4 g/mol and a molecular formula of C17H18NaN3O3S.

A “medium chain triglyceride” as used herein refers to a fatty acid triglyceride ester in which the fatty acids are generally the range of, for example, C6 to C12 (where the C followed by the number indicates the number of carbon atoms in the chain). Examples include triglyceride compositions comprising fatty acids of C6, C8, C10, and C12 in length, or mixtures of two or more of those. A “long chain triglyceride” as used herein refer to a fatty acid triglyceride ester in which the fatty acids are generally longer than C12 in length, such as C14, C16, C18, and C20 or mixtures of two or more of those. Long chain triglyceride compositions may also comprise fatty acids of C22 or C24 or C26 in length. A medium chain triglyceride composition may comprise low amounts of longer chain triglycerides, such as, for example, less than 20% or less than 10% or less than 5%. Likewise, a long chain triglyceride composition may comprise low amounts of medium chain triglycerides, such as less than 20% or less than 10%, or less than 5%. A composition comprising “medium to long chain fatty acids” may comprise a mixture of fatty acids comprising medium chain and/or long chains as described above. A composition comprising “medium to long chain triglycerides” may comprise a mixture of triglycerides comprising medium chain and/or long chain triglycerides. Examples of such compositions include a variety of plant and animal oils, as well as products derived from such oils, for example by subjecting a natural oil to various processing or purification steps designed to alter the fatty acid composition.

A “plant oil” refers to an oil derived from a plant species. Some examples include, for instance, canola oil (aka. rapeseed oil), coconut oil, corn oil, cottonseed oil, olive oil, palm oil, peanut oil, safflower oil, sesame oil, soybean oil, and sunflower oil, as well as mixtures of two or more of the above oils. It also includes oils derived from plants after processing, for example, to modify the fatty acid composition of the oil, or to remove or reduce the concentration of particular components such as shorter (e.g. C14 or lower) or longer chain (e.g. C20 or C22, or C24 or higher) fatty acids. Plant oils may in some cases contain a majority of C16 and C18 fatty acids (e.g., may comprise mostly long chain fatty acids), such as at least 80% of C16 and C18 fatty acids like palmitic, stearic, oleic, and linoleic fatty acids. “Cottonseed oil” is an oil derived from the cottonseed plant. Cottonseed oil commonly contains a mixture of palmitic, stearic, oleic, and linoleic fatty acids. Commercial preparations of cottonseed oil may contain, for example, a mixture of about 20% palmitic (C16:0), 2% stearic (C18:0), 35% oleic (C18:1), and 42% linoleic (C18:2) fatty acids.

“Caprylic/capric triglycerides” is a term used to describe a mixture of primarily C8 and C10 fatty acid triglycerides. Caprylic/capric triglycerides may, in some embodiments, include a composition comprising 50-65% caprylic (C8) and 30-45% capric (C10) acid. Caprylic/capric triglycerides may be derived from plant oils such as coconut oil and palmseed oil. Caprylic/capric triglycerides are sold commercially, for example, under the names Miglyol® 812 and Captex® 355, for example.

In referring to a particular type of fatty acid herein, such as capric, caprylic, palmitic, or oleic acid, for example, the term includes any common chemical form of the molecule such as a free fatty acid, salt of a free fatty acid, and a fatty acid ester such as a mono, di, or triglyceride, or phospholipid. For example, fatty acids found in vegetable oils are frequently in the triglyceride form. As individual triglyceride molecules contain three fatty acid chains, they may contain one, two, or three different types of fatty acid. A fatty acid analysis is commonly performed to provide the relative amounts, for example by weight percentages, of each type of fatty acid in an oil regardless of the form in which it is found in the original oil (e.g. as part of a triglyceride or other ester, and point of attachment to a triglyceride).

The fatty acids found in natural oils typically are in the triglyceride form. The compositions herein may also contain other ingredients and byproducts of the plant oils such as glycerin. The compositions may not be purely in the triglyceride form. For example, triglyceride compositions may also contain monoglycerides and diglycerides and other esterified fatty acids. They may also contain other ingredients naturally found in oils such as glycerin.

A “preservative” includes compounds that are pharmaceutically acceptable for an injectable formulation, and that may help to improve the shelf-life of the formulation. For example, preservatives may include antioxidants in some embodiments. In some embodiments, the preservative consists essentially of one or more antioxidants.

The formulations herein may be administered, for example, by intramuscular or subcutaneous injection. “Administering” more generally herein refers to the physical introduction of a composition comprising a therapeutic agent to a subject, using any of the various methods and delivery systems known to those skilled in the art. Administering can be performed, for example, once, a plurality of times, and/or over one or more extended periods.

A “pharmaceutical formulation” or “therapeutic formulation” as used herein refers to a composition comprising a therapeutic agent such as esomeprazole that is suitable for pharmaceutical use, such as, for example, administration to an animal either directly or after being reconstituted, diluted, mixed, or dissolved with at least one further composition, or thawed from a frozen state. A “ready-to-use” formulation, as used herein, means a formulation that can be administered to directly and therefore, does not need to be diluted, reconstituted, thawed from a frozen state, dissolved in solution, or mixed with other ingredients prior to administration.

In some embodiments herein, pharmaceutical formulations “do not comprise” one or more types of excipients or ingredients. The expression “does not comprise” in this context means that the excluded ingredients are not present beyond trace levels, for example, due to contamination or impurities found in other purposefully added ingredients.

The term “consisting essentially of” when referring to a mixture of ingredients of a formulation herein indicates that, while ingredients other than those expressly listed may be present, such ingredients are found only in trace amounts or in amounts otherwise low enough that the fundamental characteristics of the formulation including esomeprazole concentration, viscosity, stability upon storage, osmolality, and pH are not significantly changed. For example, ingredients such as cottonseed oil and caprylic/capric triglycerides, because they are derived from natural sources, may contain other ingredients and byproducts in small or trace quantities, while esomeprazole may also contain trace levels of various impurities from its synthesis or due to natural degradation processes.

A “vial” herein includes any bottle or container of any size or shape that is suitable for storing a pharmaceutical formulation herein. In some cases, a vial may be used not only for storage of the formulation but for drawing up a suitable volume of the formulation for injection to a subject. Vials may contain sufficient amounts to provide one or multiple doses of the formulation.

A “device” herein refers to a means of administering the formulation intramuscularly, and can be of any suitable structure and configuration that is suitable for intramuscular injection. Examples of “devices” herein include syringes equipped with needles used to deliver the contents of the syringes by intramuscular injection. Other examples include auto-injectors akin to those used to administer human drugs intramuscularly such as norepinephrine, epinephrine, adrenaline and insulin.

The term “ulcer” or “gastric ulcer” herein encompasses ulcerative disease of the stomach and gastrointestinal tract, such as the intestine, as well as suspected ulcerative disease. Ulcers, for example, may comprise open sores in the lining of the stomach or intestine. Ulcers may be diagnosed, for example, by endoscopy, or in some cases may be suspected based on phenotypic behaviors in equines commonly associated with ulcers such as irritability, discomfort, anxiety, and/or uncooperativeness that occurs particularly during riding or training, weight loss, loss of appetite, and loss of coat condition. In some embodiments, an ulcer can be equine gastric ulcer syndrome (EGUS). In some embodiments, the ulcer can be equine squamous gastric disease (ESGD), or equine glandular gastric disease (EGGD).

“Treatment,” as used herein, refers to therapeutic treatment, for example, in order to obtain a partial or complete resolution of an ulcer, slow the progression of an ulcer, or reduce its severity. The term “treatment” also includes reducing the severity of any phenotypic characteristic associated with an ulcer and/or reducing the incidence, degree, or likelihood of that characteristic, such as irritability, discomfort, anxiety, and/or uncooperativeness that occurs particularly during riding or training, weight loss, loss of appetite, and loss of coat condition. Those in need of treatment include animals already diagnosed with gastric ulcer as well as those who have previously had ulcers and are at risk of a recurrence or a worsening of phenotypic symptoms of the disorder and those who are at risk of developing ulcers (e.g. animals undergoing stress such as transport). Treatment of gastric ulcer in an animal such as an equine may involve, for example, reducing the size and/or number of gastric lesions, partially or completely resolving existing lesions, increasing the overall pH of gastric juices in the subject, as well as reducing symptoms and phenotypes commonly associated with ulcer such as poor condition (e.g. weight loss and poor coat quality), irritability, anxiety, or uncooperativeness during riding and training activities, and other phenotypic symptoms of gastric upset.

The term “effective amount” or “therapeutically effective amount” refers to an amount of a drug effective to treat an ulcer in a subject. In certain embodiments, an effective amount refers to an amount effective, at dosages and for periods of time necessary, to achieve the desired therapeutic result, such as a lessening of physical or phenotypic symptoms associated with ulcers in equines.

Additional definitions are provided in the sections that follow.

### II. Pharmaceutical Formulations and their Preparation

The present disclosure relates, among other things, to injectable pharmaceutical formulations comprising a proton pump inhibitor such as omeprazole or esomeprazole suitable for treating equines and other animals such as livestock animals. In some embodiments, the formulations comprise a suspension of the proton pump inhibitor such as omeprazole or esomeprazole in a mixture of plant oil and caprylic/capric triglyceride. In some embodiments, the proton pump inhibitor can be omeprazole, esomeprazole, rabeprazole, lansoprazole, or tenatoprazole. In some embodiments, the proton pump inhibitor can be a magnesium salt form of omeprazole, esomeprazole, rabeprazole, lansoprazole, or tenatoprazole.

In some embodiments, the formulation comprises omeprazole such as an omeprazole salt or hydrated salt. The omeprazole may be an omeprazole magnesium, or a different omeprazole form such as omeprazole sodium. In some embodiments, the formulation comprises esomeprazole such as an esomeprazole salt or hydrated salt. The esomeprazole may be an esomeprazole magnesium, or a different esomeprazole form such as esomeprazole sodium or esomeprazole strontium. In formulations comprising esomeprazole magnesium, the esomeprazole magnesium may be an esomeprazole magnesium dihydrate or trihydrate form or could be a mixture of those forms. For example, a formulation could be prepared by adding esomeprazole dihydrate, but the dihydrate could have trace amounts of trihydrate or could slowly convert to a trihydrate form over time in a suspension or upon exposure to water. Thus, for example, esomeprazole dihydrate may contain small amounts of esomeprazole trihydrate and vice versa due to impurities in the products or based on the extent to which the product has been exposed to water. The plant oil may, in some embodiments, be any one of canola oil (aka. rapeseed oil), coconut oil, corn oil, cottonseed oil, olive oil, palm oil, peanut oil, safflower oil, sesame oil, soybean oil, and sunflower oil, as well as mixtures of any two or more of the above oils. In some embodiments, the plant oil is cottonseed oil or a mixture of cottonseed oil and at least one other plant oil.

In some embodiments, the formulation comprises 15% to 25% weight/weight (w/w) proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium. In other embodiments, the formulation comprises 18% to 22% w/w proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium. In some embodiments, the formulation comprises 19% to 21% w/w proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium. In some embodiments, the formulation comprises 15%, 16%, 17%, 18%, 19%, 20%, 21%, 22%, 23%, 24%, or 25% w/w proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium. In some embodiments, the formulation comprises 20% w/w proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium. In some embodiments, the formulation comprises 15% to 25% weight/weight (w/w) esomeprazole magnesium or omeprazole magnesium. In other embodiments, the formulation comprises 18% to 22% w/w esomeprazole magnesium or omeprazole magnesium. In some embodiments, the formulation comprises 19% to 21% w/w esomeprazole magnesium or omeprazole magnesium. In some embodiments, the formulation comprises 15%, 16%, 17%, 18%, 19%, 20%, 21%, 22%, 23%, 24%, or 25% w/w esomeprazole magnesium or omeprazole magnesium. In some embodiments, the formulation comprises 20% w/w esomeprazole magnesium or omeprazole magnesium. In some embodiments, the formulation comprises an esomeprazole magnesium that is stable for pharmaceutical use for at least 36 months.

In some embodiments, the formulation comprises 5-30% plant oil, such as 5-25%, 10-15%, 10-12%, 13-15%, 15-17%, 18-20%, 15-20%, 20-22%, 23-25%, 20-25%, 20-30%, or 25-30% w/w plant oil. In some embodiments, the formulation comprises 5-25% w/w plant oil. In some embodiments, the formulation comprises 8-15% w/w plant oil. In some embodiments, the formulation comprises 10-15% w/w plant oil. In some embodiments, the formulation comprises 10-12% w/w plant oil. In some embodiments, the formulation comprises 13-15% w/w plant oil. In some embodiments, the formulation comprises 15-17% w/w plant oil. In some embodiments, the formulation comprises 18-20% w/w plant oil. In some embodiments, the formulation comprises 15-20% w/w plant oil. In some embodiments, the formulation comprises 20-22% w/w plant oil. In some embodiments, the formulation comprises 23-25% w/w plant oil. In some embodiments, the formulation comprises 20-25% w/w plant oil. In some embodiments, the formulation comprises 25-30% w/w plant oil. In some embodiments, the formulation comprises 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w plant oil.

In some embodiments, the formulation comprises 5-30% cottonseed oil, such as 5-25%, 10-15%, 10-12%, 13-15%, 15-17%, 18-20%, 15-20%, 20-22%, 23-25%, 20-25%, 20-30%, or 25-30% w/w cottonseed oil. In some embodiments, the formulation comprises 5-25% w/w cottonseed oil. In some embodiments, the formulation comprises 8-15% w/w cottonseed oil. In some embodiments, the formulation comprises 10-15% w/w cottonseed oil. In some embodiments, the formulation comprises 10-12% w/w cottonseed oil. In some embodiments, the formulation comprises 13-15% w/w cottonseed oil. In some embodiments, the formulation comprises 15-17% w/w cottonseed oil. In some embodiments, the formulation comprises 18-20% w/w cottonseed oil. In some embodiments, the formulation comprises 15-20% w/w cottonseed oil. In some embodiments, the formulation comprises 20-22% w/w cottonseed oil. In some embodiments, the formulation comprises 23-25% w/w cottonseed oil. In some embodiments, the formulation comprises 20-25% w/w cottonseed oil. In some embodiments, the formulation comprises 25-30% w/w cottonseed oil. In some embodiments, the formulation comprises 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% w/w cottonseed oil.

In some embodiments, the plant oil or the cottonseed oil may comprise at least 80% or at least 90% C16 and C18 fatty acids. In some embodiments, the plant oil or the cottonseed oil may comprise at least 80% or at least 90% C16:0 and C18:0, C18:1, and C18:2 fatty acids. In some embodiments, the plant oil or the cottonseed oil may comprise each of palmitic (C16:0) and/or stearic (C18:0), oleic (C18:1), and linoleic (C18:2) fatty acids. In some embodiments, the plant oil or the cottonseed oil may comprise each of palmitic (C16:0), stearic (C18:0), oleic (C18:1), and linoleic (C18:2) fatty acids. In some embodiments, the plant oil or cottonseed oil may contain, for example, about 15-25% palmitic, 1-5% stearic, 30-40% oleic, and 35-45% linoleic (C18:2) fatty acids. In some embodiments, the plant oil or cottonseed oil may contain, for example, a mixture of about 20% palmitic (C16:0), 2% stearic (C18:0), 35% oleic (C18:1), and 42% linoleic (C18:2) fatty acids. As plant oils such as cottonseed oils are natural products, they may also contain other oils besides those listed above as well as other ingredients, for example, as byproducts.

In some embodiments, the formulation comprises 50% to 90% w/w caprylic/capric triglyceride, such as 50-60%, 60-70%, 70-80%, or 80-90%. In some embodiments, the formulation comprises 60-70%, 60-65%, 65-70%, 70-80%, 70-75%, or 75-80% w/w caprylic/capric triglyceride. In some embodiments, the formulation comprises 50% to 60% caprylic/capric triglyceride. In some embodiments, the formulation comprises 60% to 70% caprylic/capric triglyceride. In some embodiments, the formulation comprises 60% to 65% w/w caprylic/capric triglyceride. In some embodiments, the formulation comprises 65% to 70% caprylic/capric triglyceride. In some embodiments, the formulation comprises 70% to 75% caprylic/capric triglyceride. In some embodiments, the formulation comprises 75% to 80% caprylic/capric triglyceride. In some embodiments, the formulation comprises 80% to 90% caprylic/capric triglyceride. In some embodiments, the formulation comprises 50%, 55%, 60%, 65%, 70%, 75%, 80%, 85%, or 90% caprylic/capric triglyceride.

In some embodiments, the caprylic/capric triglyceride comprises 50-65% caprylic acid and 30-45% capric acid. In some embodiments, the caprylic/capric triglyceride comprises 50-75% caprylic acid and 20-45% capric acid. In other embodiments, the mixture is 50-60% caprylic acid to 30-40% capric acid. In some embodiments, the caprylic/capric triglyceride is Miglyol® 812, Miglyol® 810, or Captex® 355. Furthermore, caprylic/capric triglycerides are commonly derived from plant or vegetable oils such as palmseed oil and coconut oil. Thus, they may contain other ingredients typically found in such oils such as glycerin or other non-fatty acid molecules, as well as other fatty acids beyond capric and caprylic acids, as well as mono- or diglycerides of capric and caprylic acid or other fatty acids. In some embodiments, the caprylic/capric triglyceride also comprises other fatty acids such as myristic acid (C14:0), lauric acid (C12:0) and caproic acid (C6:0).

As discussed above, in referring to a particular type of fatty acid herein, such as capric, caprylic, palmitic, or oleic acid, for example, or medium-chain or long-chain fatty acids, the term includes any common form of the molecule such as a free fatty acid, a salt of a fatty acid, and a fatty acid ester such as a mono, di, or triglyceride, or a phospholipid. For example, fatty acids found in plant oils are frequently in the triglyceride form. Triglycerides, particularly in natural oils, may, however, contain impurities such as mono- and diglycerides or other esteric fatty acid forms.

Additional embodiments herein comprise injectable pharmaceutical formulations comprising a suspension of esomeprazole magnesium dihydrate in an oil comprising medium-chain to long-chain fatty acids, such as medium-chain to long-chain triglycerides. In some embodiments, the median particle size of the formulations is less than 10 μm, such as less than 8 μm, less than 5 μm, less than 4 μm, or less than 2.5 μm.

In some embodiments, the medium-chain to long-chain fatty acids comprise medium-chain fatty acids. In some embodiments, the medium-chain to long-chain fatty acids comprise long-chain fatty acids. In some embodiments, they comprise a mixture of medium-chain and long-chain fatty acids. In some embodiments, the medium-chain to long-chain triglycerides comprise long-chain triglycerides. In some embodiments, they comprise a mixture of medium-chain and long-chain triglycerides.

In some embodiments, the fatty acids or triglycerides comprise or consist of one or more plant oils. In some embodiments, the plant oil is selected from the group consisting of: canola oil, coconut oil, corn oil, cottonseed oil, olive oil, palm oil, peanut oil, safflower oil, sesame oil, soybean oil, and sunflower oil, and mixtures of any two or more of canola oil, coconut oil, corn oil, cottonseed oil, olive oil, palm oil, peanut oil, safflower oil, sesame oil, soybean oil, and sunflower oil, and mixtures of any of those oils. In some embodiments, the plant oil is cottonseed oil or a mixture of cottonseed oil with another plant oil. In some embodiments, the medium-chain to long-chain triglycerides comprise caprylic/capric triglyceride or another product that is similarly derived from plant oil. In some embodiments, the caprylic/capric triglyceride comprises 50-65% caprylic acid and 30-45% capric acid, or wherein the caprylic/capric triglyceride comprises Miglyol® 812. In some embodiments, the caprylic/capric triglyceride comprises 50-75% caprylic acid and 22-45% capric acid, or wherein the caprylic/capric triglyceride comprises Captex® 355.

In some embodiments, the above formulation comprises 50% to 90% w/w caprylic/capric triglyceride, such as 50-60%, 60-70%, 70-80%, or 80-90%. In some embodiments, the formulation comprises 60-70%, 60-65%, 65-70%, 70-80%, 70-75%, or 75-80% w/w caprylic/capric triglyceride. In some embodiments, the formulation comprises 15% to 25% weight/weight (w/w) esomeprazole magnesium dihydrate. In some embodiments, the formulation comprises 18% to 22% w/w esomeprazole magnesium. In some embodiments, the formulation comprises 19% to 21% w/w esomeprazole magnesium. In some embodiments, the formulation comprises 20% w/w esomeprazole magnesium. In some embodiments, the formulation comprises 5% to 30% w/w plant oil, such as 5-10%, 10-15%, 15-20%, 20-25%, or 25-30%. In some embodiments, the formulation comprises 5% to 30% w/w cottonseed oil, such as 5-10%, 10-15%, 15-20%, 20-25%, or 25-30%. In some embodiments, the formulation comprises 10-15%, 10-12%, 13-15%, 15-17%, 18-20%, or 15-20% w/w cottonseed oil.

In some embodiments, the formulations herein comprise other excipients beyond the proton pump inhibitor, medium-chain to long-chain triglycerides, caprylic/capric triglyceride, and/or plant oil ingredients. For instance, in some embodiments, the formulations also comprise a preservative, such as butylated hydroxytoluene (BHT), butylated hydroxyanisole (BHA), sodium bisulfate, vitamin C, a bacterial growth inhibitor such as sodium nitrile, sulfur dioxide, benzoic acid, methylparaben, propylparaben, benzyl alcohol, phenol, a cresol such as m-cresol, chlorobutanol, or a mixture of two or more preservatives. In some cases, the preservative may consist essentially of an antioxidant preservative, such as BHA and/or BHT, for example. The preservative may comprise a single additional ingredient or more than one additional ingredient. In some embodiments, the preservative comprises butylated hydroxytoluene (BHT). In some embodiments, the preservative is present in an amount of 0.05-1.0% w/w. In some embodiments, the preservative is present at 0.05-0.15% w/w. In some embodiments, the preservative is present at 0.1% w/w. In some embodiments, the preservative is present at 0.1-1.0%, 0.1-0.2%, 0.1-0.5%, 0.5-1.0%, 0.05%, 0.08%, 0.09%, 0.1%, 0.11%, 0.12%, 0.13%, 0.14%, 0.15%, 0.2%, 0.3%, 0.4%, 0.5%, 0.6%, 0.7%, 0.8%, 0.9%, or 1.0% w/w. In some embodiments, the preservative comprises 0.05-1.0% w/w BHT. In some embodiments, the preservative comprises 0.05-0.15% w/w BHT. In some embodiments, the preservative comprises 0.1% w/w BHT. In some embodiments, the preservative comprises 0.1-1.0%, 0.1-0.2%, 0.1-0.5%, 0.5-1.0%, 0.05%, 0.08%, 0.09%, 0.1%, 0.11%, 0.12%, 0.13%, 0.14%, 0.15%, 0.2%, 0.3%, 0.4%, 0.5%, 0.6%, 0.7%, 0.8%, 0.9%, or 1.0% w/w BHT or BHA.

In some embodiments, particular combinations of the above ingredients may be used. For example, in some embodiments, the formulation consists essentially of proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium, plant oil such as cottonseed oil, caprylic/capric triglycerides, and a preservative such as BHT or BHA or sodium bisulfite. For example, in some embodiments, the formulation consists essentially of esomeprazole magnesium, plant oil such as cottonseed oil, caprylic/capric triglycerides, and a preservative such as BHT or BHA or sodium bisulfite. In some embodiments, the formulation consists essentially of omeprazole magnesium, plant oil such as cottonseed oil, caprylic/capric triglycerides, and a preservative such as BHT or BHA or sodium bisulfite. In some embodiments, the formulation consists essentially of esomeprazole magnesium dihydrate, plant oil such as cottonseed oil, caprylic/capric triglycerides, and a preservative such as BHT or BHA or sodium bisulfite. In some embodiments, the formulation consists essentially of esomeprazole magnesium dihydrate, medium-chain to long-chain triglycerides, and preservative. In some embodiments, the formulation does not comprise water or aqueous ingredients such as a buffer, except for possibly small amounts of aqueous components that are contaminants of the proton pump inhibitor, preservative, or oil-based excipients.

In some embodiments, the formulation has a median particle size of less than 10 μm, less than 8 μm, less than 5 μm, less than 4 μm, or less than 2.5 μm. In some such embodiments, the proton pump inhibitor is micronized before or after addition to the formulation in order to achieve this median particle size. For example, if the particular proton pump inhibitor species, such as a particular omeprazole or esomeprazole salt produces a formulation median particle size of, for example, above 10 μm, in some embodiments, it could be micronized to reduce the particle size. In other embodiments, the formulation has not been micronized, yet has a median particle size of less than 10 μm, less than 8 μm, less than 5 μm, less than 4 μm, or less than 2.5 μm. For example, certain formulations herein can achieve a median particle size of less than 10 μm, less than 8 μm, less than 5 μm, less than 4 μm, or less than 2.5 μm without micronization. In some such cases, the proton pump inhibitor species is a magnesium salt such as esomeprazole magnesium, such as esomeprazole magnesium dihydrate.

In some embodiments, the formulation comprises a suspension of 18-22% weight/weight (w/w) proton pump inhibitor such as omeprazole or esomeprazole, such as esomeprazole sodium, esomeprazole magnesium, or another omeprazole or esomeprazole salt or hydrated salt, such as a magnesium salt, in a mixture of cottonseed oil and caprylic/capric triglyceride, and 0.05-1.0% w/w preservative. In some embodiments, the formulation consists essentially of a suspension of 18-22% weight/weight (w/w) proton pump inhibitor such as omeprazole or esomeprazole, such as esomeprazole sodium, esomeprazole magnesium, or another omeprazole or esomeprazole salt or hydrated salt such as omeprazole magnesium or omeprazole sodium in a mixture of plant oil such as cottonseed oil and caprylic/capric triglyceride, and 0.05-1.0% w/w preservative. In some embodiments, the formulation comprises a suspension of 18-22% weight/weight (w/w) esomeprazole magnesium in a mixture of cottonseed oil and caprylic/capric triglyceride, and 0.05-1.0% w/w preservative. In some embodiments, the formulation consists essentially of a suspension of 18-22% weight/weight (w/w) esomeprazole magnesium in a mixture of plant oil such as cottonseed oil and caprylic/capric triglyceride, and 0.05-1.0% w/w preservative. In some such embodiments, the plant oil is present at 5-30% w/w and the caprylic/capric triglyceride is present at 50-90%. In some such embodiments, the plant oil is present at 8-15% w/w and the caprylic/capric triglyceride is present at 60-80%. In some such embodiments, the plant oil is present at 5-10% w/w and the caprylic/capric triglyceride is present at 70-85%. In some such embodiments, the plant oil is present at 10-15% w/w and the caprylic/capric triglyceride is present at 60-75%. In some such embodiments, the plant oil is present at 12-20% w/w and the caprylic/capric triglyceride is present at 55-70%. In some such embodiments, the plant oil is present at 15-20% w/w and the caprylic/capric triglyceride is present at 55-70%. In some such embodiments, the plant oil is present at 20-25% w/w and the caprylic/capric triglyceride is present at 50-65%. In some embodiments, the plant oil is any one of canola oil (aka. rapeseed oil), coconut oil, corn oil, cottonseed oil, olive oil, palm oil, peanut oil, safflower oil, sesame oil, soybean oil, and sunflower oil, as well as mixtures of any two or more of the above oils. In some embodiments, the plant oil is cottonseed oil or a mixture of cottonseed oil and at least one other plant oil.

The present disclosure also encompasses processes for preparing the pharmaceutical formulations described herein. An example process comprises first mixing plant oil and caprylic/capric triglyceride (or medium-chain to long-chain triglycerides) and then adding in the proton pump inhibitor to create a suspension. Where a preservative is added, it can be added in the first step with the oil or oil mixture, or it can be added with the proton pump inhibitor, or it can be added after the proton pump inhibitor.

### III. Storage Stability and Other Properties of the Formulations

In some embodiments, the median particle size of the formulations within 24 hours after preparing the formulations with storage at room temperature is less than 10 μm. In some embodiments, the median particle size of the formulations within 24 hours after preparing the formulations with storage at room temperature is less than 8 μm. In some embodiments, the median particle size of the formulations within 24 hours after preparing the formulations with storage at room temperature is less than 5 μm. In some embodiments, the median particle size of the formulations within 24 hours after preparing the formulations with storage at room temperature is less than 4 μm. In some embodiments, the median particle size of the formulations within 24 hours after preparing the formulations with storage at room temperature is less than 2.5 μm. In some embodiments, the median particle size of the formulations within 24 hours after preparing the formulations with storage at room temperature is less than 1 μm. The median particle size of a formulation, as referred to herein, refers to the median particle size of the proton pump inhibitor active ingredient in the formulation. This may be measured as described in the Examples below.

In some embodiments, the particle size of the 90^{th} percentile of the particle size distribution curve within 24 hours after preparing the formulations with storage at room temperature is less than 10 μm. In some embodiments, the particle size of the 90^{th} percentile of the particle size distribution curve within 24 hours after preparing the formulations with storage at room temperature is less than 8 μm. In some embodiments, the particle size of the 90^{th} percentile of the particle size distribution curve within 24 hours after preparing the formulations with storage at room temperature is less than 7 μm. In some embodiments, the particle size of the 90^{th} percentile of the particle size distribution curve within 24 hours after preparing the formulations with storage at room temperature is less than 5 μm.

In some embodiments, after 4 weeks of storage at 2-8, 25, or 30° C., the median particle size of the formulation remains less than 10 μm. In some embodiments, after 4 weeks of storage at 2-8, 25, or 30° C., the median particle size of the formulation remains less than 8 μm. In some embodiments, after 4 weeks of storage at 2-8, 25, or 30° C., the median particle size of the formulation remains less than 7 μm. In some embodiments, after 4 weeks of storage at 2-8, 25, or 30° C., the median particle size of the formulation remains less than 5 μm. In some embodiments, after 4 weeks of storage at 2-8, 25, or 30° C., the median particle size of the formulation remains less than 2.5 μm. In some embodiments, after 3 months of storage at 2-8, 25, or 30° C., the median particle size of the formulation remains less than 8 μm. In some embodiments, after 3 months of storage at 2-8, 25, or 30° C., the median particle size of the formulation remains less than 7 μm. In some embodiments, after 3 months of storage at 2-8, 25, or 30° C., the median particle size of the formulation remains less than 5 μm. In some embodiments, after 3 months of storage at 2-8, 25, or 30° C., the median particle size of the formulation remains less than 2.5 μm.

In some embodiments, after 6 months of storage at 2-8, 25, or 30° C., the median particle size of the formulation remains less than 10 μm. In some embodiments, after 6 months of storage at 2-8, 25, or 30° C., the median particle size of the formulation remains less than 8 μm. In some embodiments, after 6 months of storage at 2-8, 25, or 30° C., the median particle size of the formulation remains less than 7 μm. In some embodiments, after 6 months of storage at 2-8, 25, or 30° C., the median particle size of the formulation remains less than 5 μm. In some embodiments, after 6 months of storage at 2-8, 25, or 30° C., the median particle size of the formulation remains less than 4 μm. In some embodiments, after 6 months of storage at 2-8, 25, or 30° C., the median particle size of the formulation remains less than 2.5 μtm.

In some embodiments, after 3 months of storage at 2-8, 25, or 30° C., the particle size of the 90^{th} percentile of the particle size distribution curve remains less than 10 μm. In some embodiments, after 3 months of storage at 2-8, 25, or 30° C., the particle size of the 90^{th} percentile of the particle size distribution curve remains less than 8 μm. In some embodiments, after 3 months of storage at 2-8, 25, or 30° C., the particle size of the 90^{th} percentile of the particle size distribution curve remains less than 7 μm. In some embodiments, after 6 months of storage at 2-8, 25, or 30° C., the particle size of the 90^{th} percentile of the particle size distribution curve remains less than 6 μm.

Particle size distribution may be measured, for example, using a Malvern MASTERSIZER 2000 instrument.

Resuspendability may be measured by determining viscosity and whether there are significant changes in viscosity, e.g. increases of more than 20%, after a period of time in storage. For instance, in the Examples herein, viscosity was measured at 25° C. using a Brookfield DV3TLVCJ viscometer, as discussed further below. In some embodiments, there is no change in the resuspendability of the formulation after 4 weeks at 2-8, 25, or 30° C. In some embodiments, there is no change in the resuspendability of the formulation after 6 weeks at 2-8, 25, or 30° C. In some embodiments, there is no significant change in the viscosity of the formulation after 6 weeks storage at 2-8, 25, or 30° C. In some embodiments, there is no change in the resuspendability of the formulation after 3 months at 2-8, 25, or 30° C. In some embodiments, there is no significant change in the viscosity of the formulation after 3 months storage at 2-8, 25, or 30° C. In some embodiments, there is no change in the resuspendability of the formulation after 6 months at 2-8, 25, or 30° C. In some embodiments, there is no significant change in the viscosity of the formulation after 6 months storage at 2-8, 25, or 30° C. In some embodiments, there is no change in the resuspendability of the formulation after 12 months at 2-8, 25, or 30° C. In some embodiments, there is no significant change in the viscosity of the formulation after 12 months storage at 2-8, 25, or 30° C.

In some embodiments, the viscosity of the formulation is less than 300 cP at 25° C., measured using a Brookfield DV3% LVCJ viscometer at 25° C., as discussed in the examples below. In some embodiments, the viscosity remains below 300 cP after 6 months storage at 2-8, 25, or 30° C. In some embodiments, the viscosity remains below 300 cP after 12 months storage at 2-8 or 25° C. In some embodiments, the viscosity remains between 200 and 300 cP after 6 months storage at 2-8, 25, or 30° C. In some embodiments, the viscosity remains between 200 and 300 cP after 12 months storage at 2-8 or 25° C. In some embodiments, the viscosity remains between 225 and 300 cP after 6 months storage at 2-8, 25, or 30° C. In some embodiments, the viscosity remains between 225 and 300 cP after 12 months storage at 2-8 or 25° C. In some embodiments, the viscosity remains between 225 and 280 cP after 6 months storage at 2-8, 25, or 30° C. In some embodiments, the viscosity remains between 225 and 280 cP after 12 months storage at 2-8 or 25° C. In some embodiments, the viscosity remains between 250 and 300 cP after 6 months storage at 2-8, 25, or 30° C. In some embodiments, the viscosity remains between 250 and 300 cP after 12 months storage at 2-8 or 25° C.

In some embodiments, there is no change in the injectability of the formulation after 4 weeks at 2-8, 25, or 30° C. using a 3 mL 26 G×½″ syringe/needle. In some embodiments, there is no change in the injectability of the formulation after 6 weeks at 2-8, 25, or 30° C. using a 3 mL 26G×½″ syringe/needle. In some embodiments, there is no change in the injectability of the formulation after 3 months at room temperature (e.g. 25° C.) using a 3 mL 21 G×½″ syringe/needle. In some embodiments, there is no change in the injectability of the formulation after 6 months at room temperature (e.g. 25° C.) using a 3 mL 21 G×½″ syringe/needle. In some embodiments, there is no change in the injectability of the formulation after 3 months at room temperature (e.g. 25° C.) using a 3 mL 18 G×½″ syringe/needle. In some embodiments, there is no change in the injectability of the formulation after 6 months at room temperature (e.g. 25° C.) using a 3 mL 18 G×½″ syringe/needle.

In some embodiments, the total proton pump inhibitor (e.g. esomeprazole) impurities remain less than 0.15% after up to 6 months of storage at 2-8, 25, or 30° C. Impurities can be measured, for example, by HPLC. In the Examples below, impurities were examined using HPLC on an Agilent 1100 system with a Zorbax SB-C8, 150×4.6 mm, 3.5 μm analytical column with a mobile phase of phosphate buffer (pH 7.6) to acetonitrile (72.5:27.5 by volume) filtered through 0.8 μm nylon filter at a 1.0 mL/min flow rate at room temperature.

Accordingly, the formulations herein may have one or more of the following properties:
a. the formulation is suitable for intramuscular injection to an equine or other animal;
b. the formulation is suitable for subcutaneous injection to an equine or other animal;
c. after 6 months of storage at 2-8, 25, or 30° C., the median particle size of the formulation remains less than 8 μm;
d. after 6 months of storage at 2-8, 25, or 30° C., the median particle size of the formulation remains less than 5 μm;
e. after 6 months of storage at 2-8, 25, or 30° C., the median particle size of the formulation remains less than 4 μm;
f. after 6 months of storage at 2-8, 25, or 30° C., the median particle size of the formulation remains less than 2.5 μm;
g. after 6 months of storage at 2-8, 25, or 30° C., the particle size of the 90^{th} percentile of the particle size distribution curve remains less than 8 μm;
h. In some embodiments, there is no significant change in the viscosity of the formulation after 6 months storage at 2-8, 25, or 30° C.;
i. viscosity remains below 300 cP, between 200 and 300 cP, between 225 and 300 cP, or between 250 and 300 cP after 6 months of storage at 2-8, 25, or 30° C.;
j. there is no change in the inj ectability of the formulation after 6 months at room temperature (e.g. 25° C.) using a 3 mL 21 G×½″ syringe/needle;
k. total proton pump inhibitor impurities remain less than 0.15% after up to 6 months of storage at 2-8, 25, or 30° C.;
l. after 12 months of storage at 2-8 or 25° C., the median particle size of the formulation remains less than 8 μm;
m. after 12 months of storage at 2-8 or 25° C., the median particle size of the formulation remains less than 5 μm;
n. after 12 months of storage at 2-8 or 25° C., the median particle size of the formulation remains less than 4 μm;
o. after 12 months of storage at 2-8 or 25° C., the median particle size of the formulation remains less than 2.5 μm;
p. after 12 months of storage at 2-8 or 25° C., the particle size of the 90th percentile of the particle size distribution curve remains less than 8 μm;
q. there is no significant change in the viscosity of the formulation after 12 months storage at 2-8 or 25° C.;
r. viscosity remains below 300 cP, between 200 and 300 cP, between 225 and 300 cP, or between 250 and 300 cP after 12 months of storage at 2-8 or 25° C.;
s. there is no change in the injectability of the formulation after 12 months at room temperature (e.g. 25° C.) using a 3 mL 21 G×½″ to 18 G×½″ syringe/needle;
t. total proton pump inhibitor impurities remain less than 0.15% after up to 12 months of storage at 2-8 or 25° C.; and/or
u. the mean half-life (T_{1/2}) of the formulation when injected intramuscularly to horses ranges from 12 to 24 hours.

In some embodiments, after 24 months at 2-8° C. or 25° C. or 30° C., the median particle size of the formulation remains less than 10 μm, less than 8 μm, less than 5 μm, less than 4 μm, or less than 2.5 μm. In some embodiments, after 24 months at 2-8° C. or 25° C. or 30° C., the formulation remains stable for use.

### IV. Containers and Administration Methods

The present disclosure also encompasses vials for storing the formulation. In some embodiments, vials may also be suitable for containing the formulation to be administered. Vials may be of any shape or size appropriate for holding a veterinary pharmaceutical injectable formulation product.

The present disclosure also encompasses devices for intramuscular or subcutaneous injection to animals such as equines, which contain a suitable amount of the formulations herein for administration. In some embodiments, where one desires to administer the formulation by intramuscular injection through a syringe, the device may comprise a syringe and needle for intramuscular injection. In some embodiments, the device may be similar in structure to devices used to administer human medicines or other animal medicines intramuscularly such as epinephrine or insulin. For example, epinephrine is often administered using an auto-injection device often referred to as an injection pen. A similar pen type device may also be used here, which is suitable for intramuscular injection in equines or other animals.

In some embodiments, the device comprises a complete, single unit dosage of proton pump inhibitor, such as omeprazole or esomeprazole, such as esomeprazole magnesium, for example, for an average weight horse. In other embodiments, the device comprises more than one dose, such as two doses, three doses, or four doses.

In some embodiments, a vial or device may also be packaged with instructions for use, for example, to provide dosing instructions for equines and/or for other animals.

### V. Methods of Using Pharmaceutical Formulations

The present disclosure also encompasses methods of treating gastric ulcers in an animal, such as an equine, comprising administering an effective amount of a formulation herein intramuscularly or subcutaneously, for example, from a vial as discussed above and/or using a device as described above. In some embodiments, the formulation is an omeprazole formulation such as an omeprazole magnesium formulation. In some embodiments, the formulation is an esomeprazole formulation. In some such embodiments, the esomeprazole is esomeprazole magnesium, such as esomeprazole magnesium dihydrate. In some embodiments, the treatment methods comprising administering an effective amount of a formulation as described above to an animal such as an equine at least once per week by intramuscular or subcutaneous injection. In some embodiments, the formulation is administered over a period of 4 weeks. In some embodiments, the formulation is administered over a period of 3 weeks. In some embodiments, the formulation is administered once, twice or three times per week.

In some embodiments, where the formulation comprises esomeprazole magnesium, the formulation is administered so as to provide at least one dose of between 1.5 mg/kg and 5.0 mg/kg esomeprazole magnesium at least once per week. In some embodiments, the formulation is administered at a dose of 1.5 mg/kg, 1.75 mg/kg, 2.0 mg/kg, 2.5 mg/kg, 3.0 mg/kg, 3.5 mg/kg, 4 mg/kg, 4.5 mg/kg, or 5 mg/kg esomeprazole magnesium. In some embodiments, the formulation is administered according to one of the following regimens: (a) once per week for 3 weeks at a dose of 2.0-4.0 mg/kg; (b) once per week for 4 weeks at a dose of 2.0-4.0 mg/kg; (c) twice per week for 3 weeks at a dose of 2.0-4.0 mg/kg; (d) twice per week for 4 weeks at a dose of 2.0-4.0 mg/kg; or (e) any one of (a) to (d) followed by a reduced dose once or twice per week for at least one additional week.

In some embodiments, the first dose or the first two doses or the first week, two weeks, three weeks, or four weeks of doses are at a higher level (e.g. a “loading dose”) than subsequent doses. In some embodiments, the formulation is administered for a period longer than 4 weeks, optionally wherein the dose is reduced after a period of 4 weeks. In some embodiments, the formulation is administered for a period longer than 3 weeks, optionally wherein the dose is reduced after a period of 3 weeks. For example, in some embodiments, an initial dosage level may be selected for up to two weeks, one month, or six weeks, and then either the dosage amount or the dosage frequency may be reduced in the weeks or months that follow.

In any of the above dosing regimens, the formulation can be administered by intramuscular injection or by subcutaneous injection. In some embodiments, the formulation is administered using the vial and/or the device as described above. In some embodiments, the formulation is administered to an equine. In some embodiments, the equine suffers from gastric ulcer, equine gastric ulcer syndrome (EGUS), equine squamous gastric disease (ESGD), or equine glandular gastric disease (EGGD). In some embodiments, the gastric ulcer is treated. For example, in some embodiments at least one ulcerous lesion is resolved or reduced in size, and/or the number of lesions is reduced.

In some embodiments, the subject to be treated is an equine. In other embodiments, the subject to be treated is another animal, such as a livestock animal or working animal. In some embodiments, the other animal is a donkey, mule, pig, llama, or alpaca. In some embodiments, where a non-equine animal is treated, the esomeprazole dosage may be varied to a dosage equivalent to the dosage given to such an animal by the oral route. For example, some other animals require higher or lower proton pump inhibitor dosage per weight than do equines.

In some embodiments, the formulation is administered at a dosage equivalent to or greater than a dose that allows gastric pH in equines to be controlled for at least 3 days following the dosage, such as for 3-5 days following the dosage, or for 3 days, 4 days, or 5 days following the dosage. Gastric pH may be considered controlled if it remains greater than or equal to pH 4 more than 60% of the time.

In some embodiments, the formulation is administered at a dosage that, in clinical trials in equines, led to an improvement of the symptoms of gastric ulceration in horses corresponding to a reduction of at least 1 grade on the 0-3 gastric ulceration scoring system as follows:
0. Intact mucosal epithelium (can have reddening and/or hyperkeratosis)
1. Small single or small multifocal lesion
2. Large single or large multifocal lesion
3. Extensive (often coalescing) lesions with areas of apparent deep ulceration.

In some embodiments, the formulation is administered at a dosage that, in clinical trials in equines, led to an improvement of the symptoms of gastric ulceration in horses corresponding to a reduction of at least 1 grade on the 0-4 gastric ulceration scoring system as follows:
0. The epithelium is intact and there is no appearance of hyperkeratosis
1. The mucosa is intact, but there are areas of hyperkeratosis
2. Small, single, or multifocal lesions
3. Large, single or extensive superficial lesions
4. Extensive lesions with areas of apparent deep ulceration.

In some embodiments, the formulation is administered at a dosage that, in clinical trials in equines, resulted in both control of gastric pH as well as a reduction of at least 1 grade in the above gastric ulceration scoring system.

### EXAMPLES

### Example 1. Oil Suspensions Containing Esomeprazole Magnesium Dihydrate and Esomeprazole Sodium

Oil suspensions containing 20% esomeprazole sodium or esomeprazole magnesium dihydrate w/w were prepared in 50 gram batches using either esomeprazole magnesium dihydrate or esomeprazole sodium (FIS Chemicals, Ltd.) in a mixture of Miglyol® MCT 812 (Cremer) and super-refined cottonseed oil (Croda). The goal was to obtain homogeneous oil suspensions, one for each esomeprazole species, to verify the particle size distribution of each, and to check the homogeneity of the formulations after storage at room temperature for 24 hours.

To prepare the suspensions, the MCT 812 and cottonseed oil were first mixed together and then the esomeprazole sodium or esomeprazole magnesium was then added. The esomeprazole magnesium formulation was an off-white, viscous oil solution that appeared visually uniform, while the esomeprazole sodium formulation was a white, viscous oil solution that appeared visually uniform and thicker than the esomeprazole magnesium formulation. After 24 hours on bench top, the appearance of the esomeprazole magnesium formulation did not change while the esomeprazole sodium formulation appeared thicker. No phase separation was observed in either formulation.

After 24 hours storage, a particle size distribution (PSD) was performed using a Malvern MASTERSIZER 2000. The measuring range of the instrument is 0.02-2000 μm, with refractive index dispersant 1.375—hexanes, obscuration range 5-20%, sample/background measurement time=1, 5000 sample/background measurement snaps, and manual stirring speed 2000 rpm.

The particle size distribution for the esomeprazole magnesium formulation for the 10^{th} to 90^{th} percentile portions of the distribution curve (in μm) ranged from 0.12 to 4.75 μm (average of three measurements). The median particle size (i.e. at the 50^{th} percentile of the distribution) was 0.62 μm (average of three measurements). The particle size distribution for the esomeprazole sodium formulation for the 10th to 90^{th} percentile portions of the distribution curve ranged from 6.08 to 50.28 μm (average of three measurements), while the median particle size was 22.16 μm (average of three measurements).

Thus, the cottonseed oil—Miglyol® 812 suspension prepared with esomeprazole magnesium dihydrate has a significantly lower particle size range than the same formulation prepared with esomeprazole sodium. Furthermore, visually, the esomeprazole sodium formulation appeared thicker and more as a paste, perhaps due to large particle size.

Particle size distribution was also performed on sterilized esomeprazole magnesium dihydrate and esomeprazole sodium, both obtained from Shouguang Fukang Pharma Co. The particle size distribution for the esomeprazole magnesium for the 10^{th} to 90^{th} percentile portions of the distribution curve (in μm) ranged from 0.22 to 6.57 μm, with a median particle size of 2.01 μm (average of three measurements). The particle size distribution for the esomeprazole sodium for the 10^{th} to 90^{th} percentile portions of the distribution curve ranged from 8.60 to 36.49 μm, with a median particle size of 18.84 μm (average of three measurements).

Resuspendability was tested by measuring viscosity using a Brookfield viscometer (model no. DV3TLVCJ) at 25° C. and measuring viscosity of the formulation. A standard solution (S60 standard) with a nominal viscosity cP (mPa·s) of about 100 at 25° C. was used as a reference. The resuspendability of the esomeprazole magnesium formulation was also tested using 26 and 27 gage needles, and the formulation was found to pass through both types of needles.

The esomeprazole magnesium formulation was also stored for one month at room temperature, and no sedimentation was observed upon visual inspection. To determine sedimentation, following storage, formulations were shaken manually to disperse any sediment uniformly into a suspension and the suspension was allowed to settle under gravity at room temperature for one hour before appearance was visually checked.

### Example 2: Injectability of Formulation Vehicle and Esomeprazole Magnesium Formulation

The injectability of the esomeprazole magnesium formulation of Example 1 compared to the formulation vehicle (i.e. excipients only) was compared to determine if the oil suspension is filterable for sterilization and is injectable through a needle using a syringe. About 2 mL of the vehicle or the esomeprazole magnesium formulation was transferred into a 3 mL plastic syringe and a filter (for vehicle; 0.2 μm hydrophobic filter) or needle (for formulation; either 26 G×½″ or 27 G×½″) attached to the syringe, to extrude the contents into a small vial for visual analysis. The vehicle was easily passable with no blockage and relatively low resistance with the 26 G×½″ needle and, with the 27 G×½″ needle was passable with no blockage, and slightly greater resistance than the 26 G×½″ needle, but still able to extrude easily. With the 26 G×½″ needle, the esomeprazole magnesium formulation was passable with no blockage and relatively low resistance. With the 27 G×½″ needle, the formulation was passable with no blockage, and with slightly greater resistance than with the 26 G×½″ needle, but still able to extrude easily.

### Example 3: Stability of Esomeprazole Magnesium Formulation

The formulation as described in Example 1 was prepared for a stability study at 4 different temperatures (2-8° C., 25° C., 30° C., and 40° C.) with measurements taken at up to six weeks of storage to determine purity, particle size, resuspendability, and injection force.

Impurities were examined using HPLC on an Agilent 1100 system with a Zorbax® SB-C8, 150×4.6 mm, 3.5 μm analytical column with a mobile phase of phosphate buffer (pH 7.6) to acetonitrile (72.5:27.5 by volume) filtered through 0.8 μm nylon filter at a 1.0 mL/min flow rate at room temperature. The esomeprazole main peak in an HPLC chromatogram of esomeprazole magnesium dihydrate standard was 99.94%, while the esomeprazole main peak remained at least 99.91% after 4 weeks of storage at each temperature. Thus, total impurities remained less than 0.1% after 4 weeks at each temperature. After 6 weeks storage, total impurities at 2-8, 25, and 30° C. remained less than 0.1%, while at 40° C., total impurities were 0.12%.

Resuspentability, as measured by viscosity (see above), at each temperature showed no change after 4 weeks of storage and showed no significant change after six weeks at 2-8, 25, or 30° C. The results after 4 and 6 weeks are as shown in the table below.

| | Resuspendability | | |
|---|---|---|---|
| Temperature | T0 | 4 weeks | 6 weeks |
| 2-8° C. | Fluid off-white | No change | No change |
| 25° C. | oil formulation | No change | No change |
| 30° C. | that becomes | No change | No change |
| 40° C. | uniform with | No change | the formulation |
| | shaking | | turned thicker |
| | | | and looks |
| | | | like a paste |

Injectability results after 4 and 6 weeks are as shown in the table below. There was no change after 4 and 6 weeks at 2-8, 25, or 30° C. “N/D” medians not determined.

| | Injectability (3 mL syringe, 26 G × | | |
|---|---|---|---|
| | 1/2″ Needle (BD lot #8325442) | | |
| Temp. | T0 | 4 weeks | 6 weeks |
| 2-8° C. | The formulation | No change | No change |
| 25° C. | was passable with no | No change | No change |
| 30° C. | blockage, relatively | No change | No change |
| 40° C. | low resistance | No change | N/D |

Particle size distributions remained essentially the same after 4 weeks of storage at each temperature. Particle size 10^{th} to 90^{th} percentile ranged from about 0.2 to 7.0 μm after 4 weeks storage at all four temperatures. The median particle size remained about 1-2 μm after 4 weeks storage at all four temperatures. These results represent an average of three measurements.

The above results collectively indicate that the formulation is stable for at least 6 weeks at temperatures up to 30° C. Particle size did not materially change and the formulation remains injectable, with total impurity levels remaining below 0.1%. There was, however, a color change in the formulation at 30° C. from off-white to a slight yellow. At 40° C., the formulation becomes paste-like after 6 weeks when stored.

### Example 4: Long-term Storage of Esomeprazole Magnesium Formulations

A batch of the esomeprazole magnesium formulation of Example 1 was next prepared to test storage over a period of 3 and 6 months at 25° C., 30° C., and 40° C. Similar purity, particle size distribution, resuspendability, and injectability assays were performed as in Example 3 above. Resuspendability results are as shown in the table below.

| | Resuspendability | | |
|---|---|---|---|
| Temperature | T0 | 3 M | 6 M |
| 25° C. | Fluid off-white | No change | No change |
| | suspension, | from T0 | from 3 M |
| 30° C. | uniform with | Off-white, | No change |
| | shaking | resuspendable | from 3 M |
| | | after shaking | |
| 40° C. | | Beige-colored, | No change |
| | | slightly thicker | from 3 M |
| | | slurry, can be | |
| | | resuspended | |
| | | after shaking | |

Injectability results are as shown in the table below.

| | Injectability (3 mL syringe, 26 G × | | |
|---|---|---|---|
| | 1/2″ Needle (BD lot #8325442)) | | |
| Temp. | T0 | 3 M | 6 M |
| 25° C. | passable, | No change | No change |
| 32° C. | slight resistance, | No change | No change |
| 40° C. | no blockage | slightly higher | No change |
| | | resistance | from 3 M |
| | | (slurry) | |

Particle size ranges from TO to 6 months at each temperature are as follows. At TO, the 10^{th} to 90^{th} percentile particle size range was 0.24 to 6.32 μm with a median of 1.72 μm (average of 3 measurements). After 6 months at either 25 or 30° C., the particle size range generally narrowed such that the range from the 10^{th} to 90^{th} percentile was about 2.0 to 7.5 μm at both temperatures, with a median particle size of about 3.7-3.8 μm. At 40° C., the overall particle size range increased after 6 months to a 10^{th} to 90^{th} percentile range of about 4.0 to 18 μm.

After 6 months at either 25° C. or 30° C., total impurities as measured by HPLC remained at 0.11% or lower while impurities in the esomeprazole magnesium dihydrate standard solution were 0.07%. After 6 months storage at 40° C., total impurities measured by HPLC were 0.15%. Based on the results above, the formulation is sufficiently stable after 6 months at either 25° C. or 30° C., but 40° C. is not recommended for storage.

Long term storage of up to 6 months at 2-8° C., 25° C., and 40° C. was tested of a further formulation of esomeprazole magnesium dihydrate as described in Example 1, but additionally including 0.1% BHT (butylated hydroxytoluene; Spectrum Chemical) preservative. At T0, prior to storage, the formulation appears as an off-white oil suspension that becomes uniform with shaking. The formulation at T0 was passable through a 3 mL syringe with 21 G×½″ needle with no blockage and relatively low resistance, and was easily resuspendable with shaking. Total impurities measure 0.04% while those for an esomeprazole magnesium standard measure 0.07%. The particle size at T0 ranged from about 2.2 to 5.4 μm (10^{th} to 90^{th} percentile) with a median of 3.45 μm.

After 3 and 6 months storage at each temperature, appearance, injectability, and resuspendability results are as shown in the tables below.

Appearance:

| | Appearance | | |
|---|---|---|---|
| Temperature | T0 | 3 months | 6 months |
| 2-8° C. | Off-white oil | No change | No change |
| 25° C. | with shaking | No change | No change |
| 40° C. | suspension | dark purple | Dark brown |
| | that becomes | suspension | solid-like |
| | uniform | | |
| | with shaking | | |

Injectability:

Injectability Summary

| | Injectability (3 mL syringe, 21 G × 1/2″ Needle) | | |
|---|---|---|---|
| Temperature | T0 | 3 months | 6 months |
| 2-8° C. | The formulation | No change | No change |
| 25° C. | was passable | No change | No change |
| 40° C. | with no | N/D | N/D |
| | blockage, relatively | | |
| | low resistance | | |

ND = not done

Resuspendability:

| | Resuspendability | | |
|---|---|---|---|
| Temperature | T0 | 3 months | 6 months |
| 2-8° C. | easily | No change | No change |
| 25° C. | re-suspend | No change | No change |
| 40° C. | with shaking | very hard to | Does not |
| | | re-suspend | re-suspend |

Particle size after 3 months at either 2-8° C. or 25° C. remained in a range of about 2.0 to 5.0 μm with a median of about 3.0 to 3.5 μm, similar to at T0. Particle size at 40° C. was not determined.

After 6 months of storage at 2-8° C. and 25° C., respectively, there were 0.11% and 0.13% total impurities.

After 6 months of storage at 2-8° C. and 25° C., the viscosity of the formulation measured at 25° C. and 5 minutes (as described above) is about 250-290 cP, whereas, after 9 months of storage, the viscosity at 25° C. and 5 minutes was measured to be about 230-245 cP. Viscosity at T0 (at 25° C. and 5 minutes) was measured to be about 235-240 cP.

After 12 months of storage at 2-8° C. and 25° C., the particle size of the formulation remained in a range of about 1.5 to 5.0 μm with a median of about 2.5 to 3.0 μm, again similar to at T0. Particle size at 40° C. was not determined. There was also no change in resuspendability after 9 or 12 months of storage at 2-8° C. and 25° C. Viscosity after 12 months storage at 2-8° C. and 25° C. remained about 230-250 cP, measured as described above at 25° C. and 5 minutes in a Brookfield Ametek rheometer DV3T with an S60 standard with viscosity of about 100 cP. Total impurities remained less than 0.1% after 12 months storage at those two temperature ranges.

In conclusion, this formulation is stable at both 2-8° C. and 25° C. for 6-12 months; the appearance of the formulation did not change, and it was easily injected and resuspended. The particle size distribution as well as viscosity remained about the same as at T0, while total impurities remained less than 0.15% and only increased slightly between the two temperatures (from 0.11% to 0.13%) while those of the standard were 0.07%. At 40° C. after several months, the formulation changed color from off-white to purple and formed lumps and could not be resuspended. Thus, other tests at this temperature were not performed.

Similar 3 month stability tests were also conducted for the formulation including preservative at 30° C. After 3 months at 30° C., the appearance of the formulation did not change, it could be injected easily, did not show a significant change in impurities, but was slightly difficult to resuspend and became viscous.

### Example 5: Clinical Testing of Esomeprazole Magnesium Dihydrate Injectable Formulation

A clinical study in five horses was conducted of a suspension of 195.8 mg/mL esomeprazole magnesium dihydrate in a mixture of cottonseed oil and Miglyol® 812 excipients, including BHT preservative (antioxidant), as described in Example 4, to determine its efficacy in controlling the pH of gastric juice in the horses.

The general trial protocol was as follows. Five horses with gastric cannulas were acclimated to study conditions. Horses were healthy through the acclimation period. Baseline gastric pH measurements were recorded on prior to product administration. On Day 2, each horse was treated intramuscularly with a dose of the product intended to deliver 1.75 mg of esomeprazole magnesium dihydrate per kg bodyweight. After a washout period, the study was repeated with a dose of the product intended to deliver 4.0 mg of esomeprazole magnesium dihydrate per kg bodyweight. Gastric pH was measured following treatment.

A first 5-day treatment period evaluated the effects on gastric pH of treatment with quantities of the formulation intended to deliver 1.75 mg per kg body weight, rounded up to the next greater, 0.2 mL increment. A second 5-day treatment period tested the effects on gastric pH of quantities of the formulation intended to deliver a dosage of 4.0 mg/kg esomeprazole magnesium dihydrate.

The product was provided as a sterile suspension.

Horses were light, saddle breed horses, approximately 3 years of age, as estimated by habitus, approximately 378 to 428 kg body weight, and comprised of castrated males (geldings) and/or intact females (mares). Female candidates were not pregnant or lactating. Practices were implemented to avoid or minimize discomfort, distress, or pain for the participating animals.

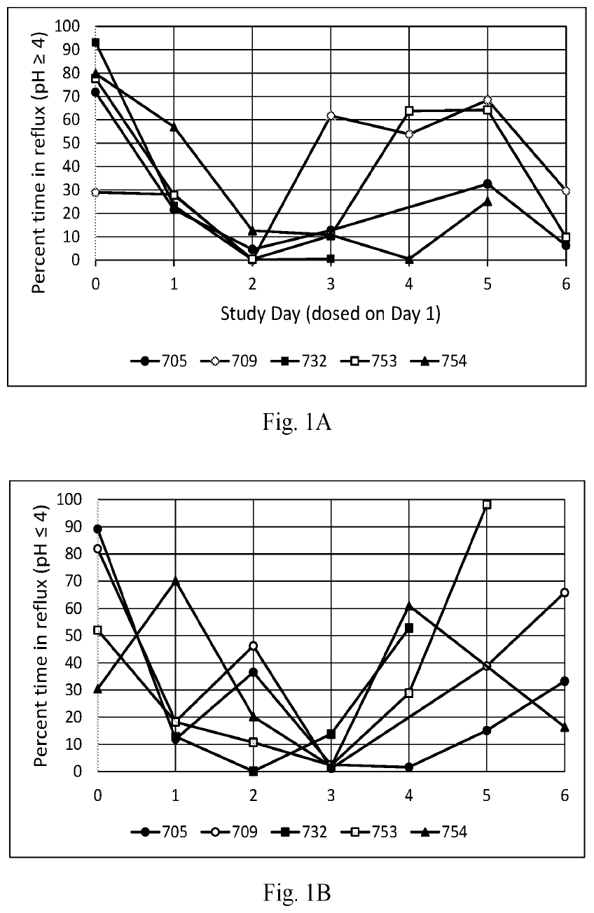

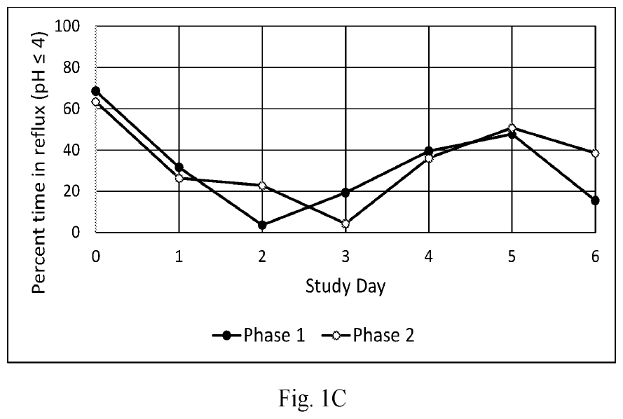

Gastric pH was measured continuously from immediately after treatment until the subsequent dose on the following day. Gastric pH was measured at baseline and following each of the two treatments at different dosages. Control of gastric pH was variable among individual horses, as expected. (See FIGS. 1A, 1B, and 1C.) The product administered at each of the 1.75 mg/kg and 4.0 mg/kg dosages controlled gastric pH for approximately 3 to 5 days in individual horses. The experimental regimen had no apparent negative impact on equine health, and appeared to be safe for intramuscular administration to mature horses.

### Example 6: A Randomized, Placebo-Controlled, Dose Ranging, Pilot Clinical Trial to Assess the Effectiveness and Field Safety of Esomeprazole Magnesium Dihydrate Injectable Formulation for the Resolution of Gastric Ulcers in Horses

A larger clinical study of a suspension of about 200 mg/mL esomeprazole magnesium dihydrate in a mixture of cottonseed oil and Miglyol® 812 excipients, including BHT preservative, as described in Example 4, was conducted on 53 horses to test effectiveness in controlling gastric ulcers at particular dosage regimens. The suspension was tested against a control saline solution.

The study was a multi-center, blinded, placebo-controlled, randomized clinical trial to investigate the effectiveness and safety of the product administered intramuscularly to control gastric ulceration in horses. The primary effectiveness endpoint was the resolution of gastric ulceration in horses, defined as a score of 0 on the 0-3 gastric ulceration scoring system. The secondary effectiveness endpoint was the improvement of gastric ulceration in horses, defined as a decrease of at least 1 grade on a 0-3 gastric ulceration scoring system as follows:
0. Intact mucosal epithelium (can have reddening and/or hyperkeratosis)
1. Small single or small multifocal lesion
2. Large single or large multifocal lesion
3. Extensive (often coalescing) lesions with areas of apparent deep ulceration.

A total of 53 horses were randomized to one of four treatment groups (4 mg/kg weekly, 2 mg/kg weekly, 2 mg/kg twice weekly, control product twice weekly). Fourteen horses were treated with 4 mg/kg investigational veterinary product (IVP) weekly, 13 horses were treated with 2 mg/kg IVP weekly, 13 horses were treated with 2 mg/kg IVP twice weekly, and 13 horses were treated with control saline (CP) twice weekly. Prohibited medicines during the study included medications that may alter gastric pH. The investigational animals were at least 1 year of age, of any sex, intact or neutered, non-pregnant, non-lactating, and non-breeding, were of any breed, and of no specified initial body weight range. All horses were determined to be evaluable for the effectiveness analysis prior to study unblinding. Of the 14 horses treated with 4 mg/kg weekly, 11 (78.6%) resolved ulcers. Of the 13 horses treated with 2 mg/kg twice weekly, 10 (76.9%) resolved ulcers. Of the 13 horses treated with 2 mg/kg weekly, 11 (84.6%) resolved ulcers. Of the horses in the placebo group, 30.8% resolved ulcers. All esomeprazole treatment groups showed statistically significant difference from the control treated group (analysis by Fisher's exact test). The product was also well tolerated.

Baseline gastroscopy examination was performed to confirm the presence of gastric ulcers, and randomization procedures were completed. Horses were administered a single dose of the IVP or CP. Gastroscopy examination and assessment was again completed at Visit 2 (Day 14). In the event the gastroscopy assessment at Visit 2 was grade 0 the horse was terminated from the study. Horses with a gastroscopy assessment at Visit 2 of grade 1 or greater continued to Visit 3. Gastroscopy assessments and study termination activities were completed on the remaining horses at Visit 3 (Day 21). Primary effectiveness was determined by a gastric ulcer resolution (gastroscopy assessment grade=0) at study termination. Secondary effectiveness was determined by a gastric ulcer improvement (gastric ulcer grade decreased by at least 1 at study termination in comparison to baseline).

The intended dose of the product was either 4 mg/kg body weight or 2 mg/kg body weight, calculated in mL based on the initial weight of the horse and the 200 mg/mL esomeprazole magnesium dihydrate concentration of the formulation, and rounded to the nearest mL. The control saline was provided at a standard volume. Doses were administered with a new 18-guage, 1.5-inch needle and 5-10 mL sterile syringe. Duration of dosing was dependent on the resolution of ulcers, and continued for a maximum of up to 18 days.

The dosing schedules per treatment group were as shown in the table below:

| Treatment | Study Day | | | | | |
|---|---|---|---|---|---|---|
| Group | Day 1 | Day 4 | Day 8 | Day 11 | Day 15 | Day 18 |
| 1 | 4 mg/kg | n/a | 4 mg/kg | n/a | 4 mg/kg | n/a |
| 2 | 2 mg/kg | 2 mg/kg | 2 mg/kg | 2 mg/kg | 2 mg/kg | 2 mg/kg |
| 3 | 2 mg/kg | n/a | 2 mg/kg | n/a | 2 mg/kg | n/a |
| 4⁽*⁾ | Matched saline | Matched saline | Matched saline | Matched saline | Matched saline | Matched saline |

⁽*⁾Matched saline was dosed at the same frequency as Group 2, but was dosed with a standard volume.

The primary measurement variable for efficacy was the resolution of ulcers (score of “0”) at study termination. The secondary measurement variable was the improvement in gastric ulcer score of at least 1 at study termination. Results were summarized by frequencies and counts. An exploratory statistical analysis of IVP to CP was conducted. Horses whose ulcers had resolved by Visit 2 were terminated at that visit. Any horses not terminated at Visit 2 were terminated at Visit 3.

The 14 horses treated with 4 mg/kg IVP were administered a total of 35 doses. Of the 14 horses treated with 4 mg/kg weekly IVP 11 (78.6%) were determined to be a treatment improvement. The 13 horses treated with 2 mg/kg IVP (either weekly or twice weekly dose groups) were administered a total of 103 doses. Of the 13 horses treated with 2 mg/kg twice weekly IVP 12 (92.3%) were determined to be a treatment improvement. Of the 13 horses treated with 2 mg/kg weekly IVP 13 (100.0%) were determined to be a treatment improvement. The 13 horses treated with CP were administered a total of 74 doses. Of the 13 horses treated with CP 7 (53.8%) were determined to be a treatment improvement. A comparison of confidence intervals showed a statistical difference between the 2 mg/kg twice weekly IVP group and the 2 mg/kg weekly IVP group when compared to CP group.

Treatment success was defined as resolution (gastric ulcer score=0) of gastric ulceration. Of the 14 horses treated with 4 mg/kg weekly IVP 11 (78.6%) were determined to be a treatment success. Of the 13 horses treated with 2 mg/kg twice weekly IVP 10 (76.9%) were determined to be a treatment success. Of the 13 horses treated with 2 mg/kg weekly IVP 11 (84.6%) were determined to be a treatment success. Of the 13 horses treated with CP 4 (30.8%) were determined to be a treatment success. The IVP formulation was therefore effective in controlling gastric ulcers within three weeks after initiation of treatment schedule in treated horses.

This clinical study in horses demonstrates that the intramuscularly administered esomeprazole magnesium dihydrate formulation is effective in controlling gastric ulcers in horses. Use of the formulation resulted in 84.6% of horses treated once weekly with 2 mg/kg esomeprazole magnesium dihydrate showing effectiveness. In this study, the once weekly, 2 mg/kg treatment was the most effective. Post-treatment abnormal clinical signs and adverse events were mild and transient, and resolved shortly after treatment. The product was generally well tolerated.

Further details of the study are provided in the tables below.

Specifications of the treated horses:

| | | 4 mg/kg | 2 mg/kg | 2 mg/kg | |
|---|---|---|---|---|---|
| | | IVP | IVP 2X | IVP | |
| | Placebo | Weekly | Weekly | Weekly | All |
| | (N = 13) | (N = 14) | (N = 13) | (N = 13) | (N = 53) |
| Age (yr) | | | | | |
| Mean (SD) | 6.6 (5.11) | 6.4 (4.83) | 5.8 (3.83) | 5.5 (3.53) | 6.1 (4.28) |
| Median | 4 | 5 | 5 | 4 | 4 |
| Min, Max | 2, 16 | 2, 15 | 2, 12 | 2, 12 | 2, 16 |
| Sex | | | | | |
| Stallion | (0.0%) | 1 (7.1%) | (0.0%) | (0.0%) | 1 (1.9%) |
| Gelding | 8 (61.5%) | 7 (50.0%) | 6 (46.2%) | 3 (23.1%) | 24 (45.3%) |
| Mare | 5 (38.5%) | 6 (42.9%) | 7 (53.8%) | 10 (76.9%) | 28 (52.8%) |
| Breed | | | | | |
| Draft- | (0.0%) | 1 (7.1%) | (0.0%) | (0.0%) | 1 (1.9%) |
| Percheron | | | | | |
| Friesian | (0.0%) | (0.0%) | 1 (7.7%) | (0.0%) | 1 (1.9%) |
| Paint Horse | (0.0%) | (0.0%) | 1 (7.7%) | (0.0%) | 1 (1.9%) |
| Pony | (0.0%) | 1 (7.1%) | (0.0%) | (0.0%) | 1 (1.9%) |
| Quarter Horse | 4 (30.8%) | 5 (35.7%) | 3 (23.1%) | 6 (46.2%) | 18 (34.0%) |
| Thoroughbred | 5 (38.5%) | 5 (35.7%) | 7 (53.8%) | 7 (53.8%) | 24 (45.3%) |
| Warmblood- | 3 (23.1%) | 1 (7.1%) | 1 (7.7%) | (0.0%) | 5 (9.4%) |
| Dutch | | | | | |
| Other | 1 (7.7%) | 1 (7.1%) | (0.0%) | (0.0%) | 2 (3.8%) |
| Baseline | | | | | |
| Weight (kg) | | | | | |
| Mean (SD) | 502.8 | 466.9 | 485.9 | 490.7 | 486.2 |
| | (46.71) | (59.56) | (48.94) | (56.60) | (53.44) |
| Median | 513 | 472.5 | 498 | 504 | 497 |
| Min, Max | 435, 592 | 347, 560 | 396, 569 | 360, 558 | 347, 592 |

Treatment success; resolution of gastric ulceration:

| | | 4 mg/kg | 2 mg/kg | 2 mg/kg |
|---|---|---|---|---|
| | | IVP | IVP 2X | IVP |
| | Placebo | Weekly | Weekly | Weekly |
| N | 13 | 14 | 13 | 13 |
| Number of | 4 (30.8%) | 11 (78.6%) | 10 (76.9%) | 11 (84.6%) |
| responders | | | | |
| (Gastric Ulcer | | | | |
| Score = 0) %) | | | | |
| Difference | | 47.8% | 46.2% | 53.8% |
| in percent | | (14.8%, | (12.2%, | (22.0%, |
| responders | | 80.8%) | 80.1%) | 85.7%) |
| vs control | | | | |
| (95% C.I.) | | | | |
| P-value | | 0.0213 | 0.0472 | 0.0154 |
| compared to | | | | |
| control (Fisher’s | | | | |
| exact test) | | | | |

Treatment improvement; improvement of gastric ulceration:

| | | 4 mg/kg | 2 mg/kg | 2 mg/kg |
|---|---|---|---|---|
| | | IVP | IVP 2X | IVP |
| | Placebo | Weekly | Weekly | Weekly |
| N | 13 | 14 | 13 | 13 |
| Number of | 7 (53.8%) | 11 (78.6%) | 12 (92.3%) | 13 (100.0%) |
| improvers | | | | |
| (Gastric Ulcer Score | | | | |
| Improvement ≥1) | | | | |
| (%) | | | | |
| Difference | | 24.7% | 38.5% | 46.2% |
| in percent | | (−9.9%, | (7.7%, | (19.1%, |
| improvers | | 59.3%) | 69.2%) | 73.3%) |
| vs. control | | | | |
| (95% C.I.) | | | | |
| P-value | | 0.2365 | 0.0730 | 0.0149 |
| compared to | | | | |
| control (Fisher’s | | | | |
| exact test) | | | | |

Time to study termination:

| | Time to Study Termination (Days) | | | |
|---|---|---|---|---|
| | | 4 mg/kg | 2 mg/kg | 2 mg/kg |
| | | IVP | IVP 2X | IVP |
| | Placebo | Weekly | Weekly | Weekly |
| N | 13 | 14 | 13 | 13 |
| Mean | 19.2 | 16.6 | 17.6 | 17.9 |
| StdDev | 2.39 | 3.69 | 3.69 | 3.45 |
| Median | 20 | 17 | 20 | 20 |
| Min | 14 | 12 | 12 | 12 |
| Max | 21 | 21 | 21 | 21 |

### Example 7: Evaluation of the Pharmacokinetic Profile of an Intramuscular Formulation

This study evaluated the pharmacokinetics of the proton pump inhibitor, esomeprazole, following multiple intramuscular doses to horses.

Eighteen adult horses were randomized into three equal treatment groups and assigned one of three doses: 2 mg/kg (1×), 6 mg/kg (3×), or 10 mg/kg (5×). Doses were administered intramuscularly. Each horse received the assigned dose three times, seven days apart. Blood samples were collected at 1, 2, 4, 6, 8, 12, 24, 36, 48, 72, 96, 120, 144 hrs after each dosing, then every 48 hours until Day 28. Esomeprazole was quantified by LC-MS/MS. Data were subjected to noncompartmental analysis.

Maximum concentrations (Cmax) of esomeprazole were reached within 1.2-2 hours with a mean half-life of 11.63-24.5 hours across all doses and administrations. Dose normalization of the Cmax and area under the curve demonstrated linear pharmacokinetics across the three dosing regimens. Following administration of the final dose in the 5× group, drug was detectable for 96±26.29 hours. A single mild, self-resolving, injection site reaction was identified in one horse in the 3× group.

This study showed that intramuscular administration of esomeprazole to horses at 1×, 3×, and 5× doses every seven days for three administrations was well tolerated. Esomeprazole was rapidly absorbed from the injection site, with a prolonged half-life of 12 to 24 hours. Linear kinetic properties were observed across the tested doses. These results support a prolonged dose interval when used in horses for the treatment of gastric ulcers, such as once weekly or longer.

What is claimed is:

1. An injectable pharmaceutical formulation, comprising a suspension of a proton pump inhibitor such as omeprazole, omeprazole magnesium, omeprazole sodium, esomeprazole, esomeprazole sodium, esomeprazole magnesium, or esomeprazole magnesium dihydrate in a mixture of a plant oil and caprylic/capric triglyceride.

2. The formulation of claim 1, wherein the plant oil is selected from the group consisting of: canola oil, coconut oil, corn oil, cottonseed oil, olive oil, palm oil, peanut oil, safflower oil, sesame oil, soybean oil, and sunflower oil, and mixtures of any two or more of canola oil, coconut oil, corn oil, cottonseed oil, olive oil, palm oil, peanut oil, safflower oil, sesame oil, soybean oil, and sunflower oil.

3. The formulation of claim 1 or 2, wherein the plant oil is cottonseed oil or a mixture of cottonseed oil with another plant oil.

4. The formulation of claim 1, 2, or 3, wherein the plant oil is cottonseed oil.

5. The formulation of any one of claims 1-4, wherein the formulation comprises 15% to 25% weight/weight (w/w) esomeprazole magnesium.

6. The formulation of claim 5, wherein the formulation comprises 18% to 22% w/w esomeprazole magnesium.

7. The formulation of claim 6, wherein the formulation comprises 19% to 21% w/w esomeprazole magnesium.

8. The formulation of claim 7, wherein the formulation comprises 20% w/w esomeprazole magnesium.

9. The formulation of any one of claims 1-8, wherein the proton pump inhibitor is esomeprazole magnesium dihydrate.

10. The formulation of any one of claims 1-9, wherein the formulation comprises 5% to 30% w/w plant oil, such as 5-10%, 10-15%, 15-20%, 20-25%, or 25-30%.

11. The formulation of any one of claims 1-8, wherein the formulation comprises 5% to 30% w/w cottonseed oil, such as 5-10%, 10-15%, 15-20%, 20-25%, or 25-30%.

12. The formulation of claim 11, wherein the formulation comprises 10-15%, 10-12%, 13-15%, 15-17%, 18-20%, or 15-20% w/w cottonseed oil.

13. The formulation of any one of claims 1-12, wherein the formulation comprises 50% to 90% w/w caprylic/capric triglyceride, such as 50-60%, 60-70%, 70-80%, or 80-90%.

14. The formulation of claim 13, wherein the formulation comprises 60-70%, 60-65%, 65-70%, 70-80%, 70-75%, or 75-80% w/w caprylic/capric triglyceride.

15. The formulation of any one of claims 1-13, further comprising at least one preservative, such as butylated hydroxytoluene (BHT) or butylated hydroxyanisole (BHA) or sodium bisulfite, or a mixture of BHT, BHA and/or sodium bisulfite.

16. The formulation of claim 14, wherein the at least one preservative comprises 0.05-1.0% w/w butylated hydroxytoluene (BHT).

17. The formulation of any one of claims 1-16, wherein the formulation consists essentially of (a) the proton pump inhibitor such as omeprazole, esomeprazole, omeprazole magnesium, omeprazole sodium, esomeprazole sodium, esomeprazole magnesium, or esomeprazole magnesium dihydrate, (b) cottonseed oil, (c) caprylic/capric triglyceride, and (d) at least one preservative.

18. The formulation of claim 17, wherein the formulation consists essentially of esomeprazole magnesium such as esomeprazole magnesium dihydrate, cottonseed oil, caprylic/capric triglyceride, and at least one preservative such as BHT.

19. An injectable pharmaceutical formulation, comprising a suspension of 18-22% weight/weight (w/w) proton pump inhibitor such as omeprazole, esomeprazole, omeprazole magnesium, omeprazole sodium, esomeprazole sodium, esomeprazole magnesium, or esomeprazole magnesium dihydrate in a mixture of cottonseed oil and caprylic/capric triglyceride, and 0.05-1.0% w/w preservative.

20. An injectable pharmaceutical formulation, consisting essentially of a suspension of 18-22% weight/weight (w/w) proton pump inhibitor such as omeprazole, omeprazole magnesium, omeprazole sodium, esomeprazole, esomeprazole sodium, esomeprazole magnesium, or esomeprazole magnesium dihydrate in a mixture of cottonseed oil and caprylic/capric triglyceride, and 0.05-1.0% w/w preservative.

21. The formulation of claim 19 or 20, wherein the proton pump inhibitor is esomeprazole magnesium or esomeprazole magnesium dihydrate.

22. The formulation of claim 19, 20, or 21, wherein the formulation comprises 5-30% cottonseed oil, such as 5-25%, 10-15%, 10-12%, 13-15%, 15-17%, 18-20%, 15-20%, 20-22%, or 20-25% w/w cottonseed oil.

23. The formulation of any one of claims 19-22, wherein the formulation comprises 50% to 90% w/w caprylic/capric triglyceride, such as 60-70%, 60-65%, 65-70%, 70-80%, 70-75%, 75-80%, 80-90%, 80-85%, or 85-90%.

24. The formulation of any one of claims 1-23, wherein the caprylic/capric triglyceride comprises 50-65% caprylic acid and 30-45% capric acid, or wherein the caprylic/capric triglyceride comprises Miglyol® 812.

25. The formulation of any one of claims 1-23, wherein the caprylic/capric triglyceride comprises 50-75% caprylic acid and 22-45% capric acid, or wherein the caprylic/capric triglyceride comprises Captex® 355.

26. The formulation of any one of claims 1-25, wherein the formulation has a median particle size of less than 10 μm, less than 8 μm, less than 5 μm, less than 4 μm, or less than 2.5 μm.

27. The formulation of any one of claims 1-26, wherein the formulation has at least one of the following properties:
a) the formulation is suitable for intramuscular injection to an equine or other animal;
b) the formulation is suitable for subcutaneous injection to an equine or other animal;
c) after 6 months of storage at 2-8, 25, or 30° C., the median particle size of the formulation remains less than 8 μm;
d) after 6 months of storage at 2-8, 25, or 30° C., the median particle size of the formulation remains less than 5 μm;
e) after 6 months of storage at 2-8, 25, or 30° C., the median particle size of the formulation remains less than 4 μm;
f) after 6 months of storage at 2-8, 25, or 30° C., the median particle size of the formulation remains less than 2.5 μm;
g) after 6 months of storage at 2-8, 25, or 30° C., the particle size of the 90^{th} percentile of the particle size distribution curve remains less than 8 μm;
h) there is no significant change in the viscosity of the formulation after 6 months storage at 2-8, 25, or 30° C.;
i) viscosity remains below 300 cP, between 200 and 300 cP, between 225 and 300 cP, or between 250 and 300 cP;
j) there is no change in the injectability of the formulation after 6 months at room temperature (e.g. 25° C.) using a 3 mL 21 G×½″ to 18 G×½″ syringe/needle; and/or
k) total proton pump inhibitor impurities remain less than 0.15% after up to 6 months of storage at 2-8, 25, or 30° C.

28. The formulation of any one of claims 1-27, wherein the formulation has at least one of the following properties:
a) after 12 months of storage at 2-8 or 25° C., the median particle size of the formulation remains less than 8 μm;
b) after 12 months of storage at 2-8 or 25° C., the median particle size of the formulation remains less than 5 μm;
c) after 12 months of storage at 2-8 or 25° C., the median particle size of the formulation remains less than 4 μm;
d) after 12 months of storage at 2-8 or 25° C., the median particle size of the formulation remains less than 2.5 μm;
e) after 12 months of storage at 2-8 or 25° C., the particle size of the 90^{th} percentile of the particle size distribution curve remains less than 8 μm;
f) there is no significant change in the viscosity of the formulation after 12 months storage at 2-8 or 25° C.;
g) viscosity remains below 300 cP, between 200 and 300 cP, between 225 and 300 cP, or between 250 and 300 cP after 12 months of storage at 2-8 or 25° C.;
h) there is no change in the injectability of the formulation after 12 months at room temperature (e.g. 25° C.) using a 3 mL 21 G×½″ to 18 G×½″ syringe/needle; and/or
i) total proton pump inhibitor impurities remain less than 0.15% after up to 12 months of storage at 2-8 or 25° C.

29. An injectable pharmaceutical formulation, comprising a suspension of a proton pump inhibitor such as omeprazole, esomeprazole, omeprazole magnesium, omeprazole sodium, esomeprazole sodium, esomeprazole magnesium, or esomeprazole magnesium dihydrate in medium-chain to long-chain triglycerides, wherein the median particle size of the formulation is less than 10 μm, less than 8 μm, less than 5 μm, less than 4 μm, or less than 2.5 μm.

30. The formulation of claim 29, wherein the proton pump inhibitor is esomeprazole magnesium.

31. The formulation of claim 30, wherein the esomeprazole magnesium is esomeprazole magnesium dihydrate.

32. The formulation of any one of claims 29-31, wherein the medium-chain to long-chain triglycerides comprise plant oil selected from the group consisting of: canola oil, coconut oil, corn oil, cottonseed oil, olive oil, palm oil, peanut oil, safflower oil, sesame oil, soybean oil, and sunflower oil, and mixtures of any two or more of canola oil, coconut oil, corn oil, cottonseed oil, olive oil, palm oil, peanut oil, safflower oil, sesame oil, soybean oil, and sunflower oil.

33. The formulation of any one of claims 29-32, wherein the plant oil is cottonseed oil or a mixture of cottonseed oil with another plant oil.

34. The formulation of any one of claims 29-33, wherein the medium-chain to long-chain triglycerides comprise caprylic/capric triglyceride.

35. The formulation of claim 34, wherein the caprylic/capric triglyceride comprises 50-65% caprylic acid and 30-45% capric acid, or wherein the caprylic/capric triglyceride comprises Miglyol® 812.

36. The formulation of claim 34, wherein the caprylic/capric triglyceride comprises 50-75% caprylic acid and 22-45% capric acid, or wherein the caprylic/capric triglyceride comprises Captex® 355.

37. The formulation of any one of claims 34-36, wherein the formulation comprises 50% to 90% w/w caprylic/capric triglyceride, such as 50-60%, 60-70%, 70-80%, or 80-90%.

38. The formulation of claim 37, wherein the formulation comprises 60-70%, 60-65%, 65-70%, 70-80%, 70-75%, or 75-80% w/w caprylic/capric triglyceride.

39. The formulation of any one of claims 29-38, wherein the formulation comprises 15% to 25% weight/weight (w/w) esomeprazole magnesium dihydrate.

40. The formulation of claim 39, wherein the formulation comprises 18% to 22% w/w esomeprazole magnesium dihydrate.

41. The formulation of claim 40, wherein the formulation comprises 19% to 21% w/w esomeprazole magnesium dihydrate.

42. The formulation of claim 41, wherein the formulation comprises 20% w/w esomeprazole magnesium dihydrate.

43. The formulation of any one of claims 29-42, wherein the formulation comprises 5% to 30% w/w plant oil, such as 5-10%, 10-15%, 15-20%, 20-25%, or 25-30%.

44. The formulation of any one of claims 29-42, wherein the formulation comprises 5% to 30% w/w cottonseed oil, such as 5-10%, 10-15%, 15-20%, 20-25%, or 25-30%.

45. The formulation of claim 44, wherein the formulation comprises 10-15%, 10-12%, 13-15%, 15-17%, 18-20%, or 15-20% w/w cottonseed oil.

46. The formulation of any one of claims 29-45, further comprising at least one preservative, such as butylated hydroxytoluene (BHT) or butylated hydroxyanisole (BHA) or sodium bisulfite, or a mixture of BHT, BHA and/or sodium bisulfite.

47. The formulation of claim 46, wherein the at least one preservative comprises 0.05-1.0% w/w butylated hydroxytoluene (BHT).

48. The formulation of claim 46 or 47, wherein the formulation consists essentially of the proton pump inhibitor, medium-chain to long-chain triglycerides, and preservative.

49. The formulation of any one of claims 29-48, wherein the formulation has at least one of the following properties:
a) the formulation is suitable for intramuscular injection to an equine or other animal;
b) the formulation is suitable for subcutaneous injection to an equine or other animal;
c) after 6 months of storage at 2-8, 25, or 30° C., the median particle size of the formulation remains less than 8 μm;
d) after 6 months of storage at 2-8, 25, or 30° C., the median particle size of the formulation remains less than 5 μm;
e) after 6 months of storage at 2-8, 25, or 30° C., the median particle size of the formulation remains less than 4 μm;
f) after 6 months of storage at 2-8, 25, or 30° C., the median particle size of the formulation remains less than 2.5 μm;
g) after 6 months of storage at 2-8, 25, or 30° C., the particle size of the 90^{th} percentile of the particle size distribution curve remains less than 8 μm;
h) there is no significant change in the viscosity of the formulation after 6 months storage at 2-8, 25, or 30° C.;
i) there is no change in the injectability of the formulation after 6 months at room temperature (e.g. 25° C.) using a 3 mL 21 G×½″ to 18 G×½″ syringe/needle; and/or
j) total proton pump inhibitor impurities remain less than 0.15% after up to 6 months of storage at 2-8, 25, or 30° C.

50. A vial comprising the formulation of any one of claims 1-49.

51. A device for intramuscular injection to equines, comprising the formulation of any one of claims 1-49.

52. A process for preparing the injectable pharmaceutical formulation of any one of claims 1-28, comprising (a) mixing the plant oil or cottonseed oil with the caprylic/capric triglyceride, and (b) adding proton pump inhibitor to the mixture of (a) to create a suspension of the proton pump inhibitor in the mixture.

53. The process of claim 52, further comprising adding at least one preservative to the mixture of plant oil or cottonseed oil and caprylic/capric triglyceride and/or to the suspension.

54. A process for preparing the injectable pharmaceutical formulation of any one of claims 29-49, comprising (a) obtaining and optionally mixing the medium-chain to long-chain triglycerides, and (b) adding proton pump inhibitor to the product of (a) to create a suspension of the proton pump inhibitor in the mixture.

55. The process of claim 54, further comprising adding at least one preservative to the product of (a) and/or to the suspension.

56. A method of treating gastric ulcer in an equine, comprising administering an effective amount of the formulation of any one of claims 1-49 to the equine at least once per week by intramuscular or subcutaneous injection.

57. The method of claim 53, wherein the formulation is administered so as to provide at least one dose of between 1.5 mg/kg and 5.0 mg/kg proton pump inhibitor at least once per week.

58. The method of claim 57, wherein the formulation is administered so as to provide at least one dose of between 1.5 mg/kg and 5.0 mg/kg esomeprazole magnesium at least once per week.

59. The method of any one of claims 56-58, wherein the formulation is administered once, twice or three times per week.

60. The method of claim 59, wherein the formulation is administered once per week.

61. The method of claim 59, wherein the formulation is administered twice per week.

62. The method of claim 59, wherein the formulation is administered three times per week.

63. The method of any one of claims 56-62, wherein the formulation is administered at a dose of 1.5 mg/kg, 1.75 mg/kg, 2.0 mg/kg, 2.5 mg/kg, 3.0 mg/kg, 3.5 mg/kg, 4 mg/kg, 4.5 mg/kg, or 5 mg/kg proton pump inhibitor.

64. The method of any one of claims 56-63, wherein the formulation is administered over a period of 4 weeks.

65. The method of any one of claims 56-63, wherein the formulation is administered over a period of 3 weeks.

66. The method of any one of claims 56-65, wherein the formulation is administered according to one of the following regimens:
a. Once per week for 3 weeks at a dose of 2.0-4.0 mg/kg;
b. Once per week for 4 weeks at a dose of 2.0-4.0 mg/kg;
c. Twice per week for 3 weeks at a dose of 2.0-4.0 mg/kg;
d. Twice per week for 4 weeks at a dose of 2.0-4.0 mg/kg; or
e. The regimen of any one of (a) to (d) followed by a reduced dose once or twice per week for at least one additional week.

67. The method of any one of claims 56-65, wherein the formulation is administered for a period longer than 4 weeks, optionally wherein the dose is reduced after a period of 4 weeks.

68. The method of any one of claims 56-65, wherein the formulation is administered for a period longer than 3 weeks, optionally wherein the dose is reduced after a period of 3 weeks.

69. The method of any one of claims 56-68, wherein the formulation is administered by intramuscular injection.

70. The method of any one of claims 56-68, wherein the formulation is administered by subcutaneous injection.

71. The method of any one of claims 56-70, wherein the formulation is administered using the vial of claim 50 and/or the device of claim 51.

72. The method of any one of claims 56-71, wherein the equine suffers from equine gastric ulcer syndrome (EGUS), equine squamous gastric disease (ESGD), or equine glandular gastric disease (EGGD).

73. The method of any one of claims 56-72, wherein the ulcer is treated, such as by (a) reducing the size of at least one ulcerous lesion or (b) reducing the number of ulcerous lesions.

74. The injectable pharmaceutical formulation of any one of claims 1-49 for use in a method of treatment of ulcer in an equine, wherein the formulation is administered to the equine by intramuscular or subcutaneous injection.

75. The formulation for use of claim 74, wherein the formulation is administered so as to provide at least one dose of between 1.5 mg/kg and 5.0 mg/kg proton pump inhibitor at least once per week.

76. The formulation for use of claim 74, wherein the formulation is administered so as to provide at least one dose of between 1.5 mg/kg and 5.0 mg/kg esomeprazole magnesium at least once per week.

77. The formulation for use of claim 74 or 76, wherein the formulation is administered once, twice or three times per week.

78. The formulation for use of claim 77, wherein the formulation is administered once per week.

79. The formulation for use of claim 77, wherein the formulation is administered twice per week.

80. The formulation for use of claim 77, wherein the formulation is administered three times per week.

81. The formulation for use of any one of claims 74-80, wherein the formulation is administered at a dose of 1.5 mg/kg, 1.75 mg/kg, 2.0 mg/kg, 2.5 mg/kg, 3.0 mg/kg, 3.5 mg/kg, 4 mg/kg, 4.5 mg/kg, or 5 mg/kg esomeprazole magnesium.

82. The formulation for use of any one of claims 74-81, wherein the formulation is administered over a period of 4 weeks.

83. The formulation for use of any one of claims 74-81, wherein the formulation is administered over a period of 3 weeks.

84. The formulation for use of any one of claims 74-83, wherein the formulation is administered according to one of the following regimens:
a. Once per week for 3 weeks at a dose of 2.0-4.0 mg/kg;
b. Once per week for 4 weeks at a dose of 2.0-4.0 mg/kg;
c. Twice per week for 3 weeks at a dose of 2.0-4.0 mg/kg;
d. Twice per week for 4 weeks at a dose of 2.0-4.0 mg/kg; or
e. The regimen of any one of (a) to (d) followed by a reduced dose once or twice per week for at least one additional week.

85. The formulation for use of any one of claims 74-83, wherein the formulation is administered for a period longer than 4 weeks, optionally wherein the dose is reduced after a period of 4 weeks.

86. The formulation for use of any one of claims 74-83, wherein the formulation is administered for a period longer than 3 weeks, optionally wherein the dose is reduced after a period of 3 weeks.

87. The formulation for use of any one of claims 74-84, wherein the formulation is administered by intramuscular injection.

88. The formulation for use of any one of claims 74-84, wherein the formulation is administered by subcutaneous injection.

89. The formulation for use of any one of claims 74-88, wherein the formulation is administered using the vial of claim 50 and/or the device of claim 51.

90. The formulation for use of any one of claims 74-89, wherein the equine suffers from equine gastric ulcer syndrome (EGUS), equine squamous gastric disease (ESGD), or equine glandular gastric disease (EGGD).

91. The formulation for use of any one of claims 74-90, wherein the ulcer is treated, such as by (a) reducing the size of at least one ulcerous lesion or (b) reducing the number of ulcerous lesions.
